(19) United States
(12) Patent Application Publication
(10) Pub. No.: US 2012/0321557 A1
(43) Pub. Date: Dec. 20, 2012

(54) ANTI-ICAM3 ANTIBODY AND USE THEREOF

(75) Inventor: Naoki Kimura, Meguro-ku (JP)

(73) Assignee: FORERUNNER PHARMA RESEARCH CO., LTD., Meguro-ku, Tokyo (JP)

(21) Appl. No.: 13/581,175

(22) PCT Filed: Feb. 25, 2011
(86) PCT No.: PCT/JP2011/054368

§ 371 Date: Aug. 24, 2012

(30) Foreign Application Priority Data
Feb. 26, 2010 (JP) 2010-041547

(51) Int. Cl. C07K 16/30; A61K 51/10; G01N 33/574; C07H 21/04; G01N 21/64

(52) U.S. Cl. 424/1.49; 536/23.53; 435/6.11; 435/7.23; 530/389.7

(57) ABSTRACT

Based on an anti-ICAM3 antibody obtained by immunizing a mouse with BaF3 cells having artificially over-expressed ICAM3, a chimeric antibody has been successfully prepared which exerts cytotoxic actions, including both a proliferation-suppressing activity and an ADCC activity, on a blood cancer cell line, and which exerts a tumor regression activity in vivo.

### TECHNICAL FIELD

The present invention relates to an anti-ICAM3 antibody and use thereof. More specifically, the present invention relates to an anti-ICAM3 antibody having a cytotoxic activity and a pharmaceutical use and other uses of the anti-ICAM3 antibody.

### BACKGROUND ART

ICAMs (interacellular adhesion molecules) are glycoproteins belonging to the immunoglobulin superfamily having several extracellular immunoglobulin-like C-type domains. So far, ICAM1 to ICAM5 have been identified. ICAM3 (CD50) is a member of the ICAM family and has five immunoglobulin-like C-type domains (NPLs 1 to 3). ICAM3 has a structure very similar to those of other ICAM family molecules ICAM1 and ICAM2. While the other ICAM family molecules show a relatively ubiquitous expression distribution such as in endothelium, ICAM3 shows a unique expression pattern confined to blood cells such as lymphocytes, leukocytes, and thymocytes (NPL 1). Similarly to ICAM1 and ICAM2, ICAM3 binds to LFA-1 (lymphocyte function antigen 1; CD11a/CD18). Besides, ICAM3 binds to CD11d/CD18, and is thought to function as an accessory molecule in the immune response (NPLs 1 to 4).

ICAM3 functions as a signaling molecule, which transmits various kinds of information from the outside into the inside of the cell. Particularly, it is reported that the cross-linking with an antibody suppresses the proliferation and induces apoptosis of some blood cells. This suggests that controlling apoptosis of blood cells in vivo is also a function of ICAM3.

For example, it is reported that an anti-ICAM3 antibody suppresses the effect of promoting T cell proliferation by PMA stimulation or the like (NPL 5). Moreover, it is reported that cross-linking ICAM3 in a human thymocyte with an anti-ICAM3 antibody induces the apoptosis while increasing intracellular calcium concentration (NPL 6). Further, it is reported that apoptosis is induced also in blood cells prepared from normal human bone marrow and leukemic cell line (U937, Jurkat) (NPL 7). Furthermore, another group has reported that anti-ICAM3 antibody induces apoptosis of granulocytes, particularly basophils and neutrophils, also (NPL 8).

However, the antibodies reported so far cannot be said to have high apoptosis-inducing activity and cell-proliferation-suppressing activity. These antibodies are therefore thought to be insufficient to exert an anti-tumor effect in vivo.

### CITATION LIST

### Non Patent Literature

[NPL 1] Vazeux R et al. Nature. 1992, 360, 485-488

[NPL 2] Fawcett J et al. Nature. 1992, 360, 481-484

[NPL 3] de Fougerolles & Spronger. J. Exp. Med. 1992, 175, 185-190

[NPL 4] Van der Vieren M et al. Immunity. 1995, 3, 683-690

[NPL 5] Green J. & Thompson C. Cell Immunology. 1996. 171. 126-131

[NPL 6] Martinez-Caceres E et al. 1996. 48. 626-635

[NPL 7] Stucki A et al. Br J Haematol. 2000. 108. 157-166

[NPL 8] Kessel J et al. J Allergy Clin Immunol. 2006. 118. 831-836

### SUMMARY OF INVENTION

### Technical Problem

The present invention has been made in view of the above-described problems in the conventional techniques. An object of the present invention is to provide an antibody capable of exerting a high anti-tumor effect in vivo.

### Solution to Problem

In order to achieve the above object, the present inventors have attempted to produce an anti-ICAM3 antibody having a high proliferation-suppressing action. First, BaF3 cells having artificially overexpressed ICAM3 were established and used for immunization of a mouse to produce an anti-ICAM3 antibody. Next, the hybridoma cells were screened for based only on cell-proliferation-suppressing activity. A monoclonal antibody IB23 having a high proliferation-suppressing activity was selected therefrom. The selected mouse monoclonal antibody IB23 bound to the surfaces of various cancer cell lines, but not to the family molecules having a high homology with ICAM3, thus showing ICAM3-specific binding. Further, the present inventors determined the sequences of the heavy chain variable region and light chain variable region of the mouse monoclonal antibody IB23, followed by modification to a human chimeric antibody. Then, the in vitro cytotoxic activity of this antibody was analyzed. As a result, it was revealed that the IB23 human chimeric antibody alone exhibited a high proliferation-suppressing activity on various blood cancer cell lines. Moreover, the ADCC activity was also analyzed. As a result, it was found out that the human chimeric antibody had a high ADCC activity. In other words, the IB23 antibody was revealed as an antibody which exerts cytotoxic actions, including both a proliferation-suppressing activity and an ADCC activity, on blood cancer cell lines.

Next, whether or not the IB23 antibody exhibits an in vivo tumor regression activity was analyzed. As a result, it was found out that the IB23 human chimeric antibody showed a significant tumor regression effect on a human leukemia mouse model. A tumor regression effect was also observed from a deglycosylated IB23 antibody which had lost the ADCC activity by deglycosylation. Nevertheless, the drug action of the IB23 human chimeric antibody having both a proliferation-suppressing activity and an ADCC activity was superior to that of the deglycosylated antibody. This proved that the drug action in vivo was successfully enhanced by adding an ADCC activity to a proliferation-suppressing activity of the antibody.

Specifically, the present invention relates to an anti-ICAM3 antibody having a cytotoxic activity and a pharmaceutical use and other uses of the anti-ICAM3 antibody. More specifically, the present invention provides the following inventions.
(1) An anti-ICAM3 antibody having a cytotoxic activity.
(2) The anti-ICAM3 antibody according to (1), wherein the cytotoxic activity is an ADCC activity.
(3) The anti-ICAM3 antibody according to any one of (1) and (2), which further has a cell-proliferation-suppressing activity.
(4) The anti-ICAM3 antibody according to any one of (1) to (3), which has an anti-tumor activity in vivo.
(5) The anti-ICAM3 antibody according to any one of (1) to (4), which does not substantially bind to ICAM1 and/or ICAM5.
(6) An antibody of any one of (a) to (c) below:
   (a) an antibody comprising heavy chain CDR1 having an amino acid sequence of SEQ ID NO: 15, heavy chain CDR2 having an amino acid sequence of SEQ ID NO: 16, heavy chain CDR3 having an amino acid sequence of SEQ ID NO: 17, light chain CDR1 having an amino acid sequence of SEQ ID NO: 22, light chain CDR2 having an amino acid sequence of SEQ ID NO: 23, and light chain CDR3 having an amino acid sequence of SEQ ID NO: 24;
   (b) an antibody of (a) in which one or more amino acids are substituted, deleted, added and/or inserted, the antibody of (b) having an activity equivalent to that of the antibody of (a); and
   (c) an antibody which recognizes a same epitope as an epitope recognized by the antibody of any one of (a) and (b).
(7) The anti-ICAM3 antibody according to any one of (1) to (6), comprising a human-derived constant region.
(8) A DNA encoding the antibody according to any one of (1) to (7).
(9) A pharmaceutical composition comprising the antibody according to any one of (1) to (7) as an active ingredient.
(10) The pharmaceutical composition according to (9), which is an anti-cancer agent.
(11) The pharmaceutical composition according to (10), wherein the cancer is leukemia, myeloma, or malignant lymphoma.
(12) A diagnosis method for a cancer, characterized by comprising the step of detecting anyone of an ICAM3 protein and a gene encoding the ICAM3 protein.

### Advantageous Effects of Invention

An antibody of the present invention binds to a cell having ICAM3 expressed on the surface thereof, and shows a high cell-proliferation action and a high antibody-dependent cell-mediated cytotoxic action. Particularly, when acting on tumor cells, the antibody of the present invention demonstrates an excellent anti-tumor effect in vitro and in vivo. Thus, the antibody of the present invention makes it possible to effectively treat ICAM3-involved diseases (particularly, cancers).

### BRIEF DESCRIPTION OF DRAWINGS

FIG. 1 is a graph showing the result of detecting the cell-proliferation-suppressing activities of mouse anti-ICAM3 antibodies on KMS-12-BM cells.

FIG. 2 is a graph showing the result of detecting the binding activity of an IB23 antibody on various blood cancer lines.

FIG. 3 is graphs showing the result of detecting the binding activity of the IB23 antibody on an ICAM1-expressing BaF3 transfectant (ICAM1/BaF3) and on ICAM5-expressing BaF3 transfectant (ICAM5/BaF3).

FIG. 4 is graphs showing the result of detecting the proliferation-suppressing activity of an IB23 human chimeric antibody on various blood cancer lines.

FIG. 5 is a graph showing the result of detecting the cell-proliferation-suppressing activities of the IB23 human chimeric antibody (IB23 standard antibody) and a deglycosylated IB23 human chimeric antibody on SKM-1 cells.

FIG. 6 is graphs showing the result of detecting the ADCC activity of the IB23 human chimeric antibody (IB23 standard antibody) on an ICAM3-expressing BaF3 transfectant (ICAM3/BaF3) and various blood cancer lines.

FIG. 7 is a graph showing the result of detecting the ADCC activities of the IB23human chimeric antibody (IB23 standard antibody) and the deglycosylated IB23human chimeric antibody on an ICAM3-expressing BaF3 transfectant (ICAM3/BaF3).

FIG. 8A is a graph showing the result of detecting the in vivo anti-tumor activity of the IB23 human chimeric antibody (IB23 standard antibody).

FIG. 8B is a graph showing the result of detecting the in vivo anti-tumor activity of the deglycosylated IB23human chimeric antibody.

### DESCRIPTION OF EMBODIMENTS

[ICAM3]

“ICAM3 (interacellular adhesion molecule 3)” targeted by an antibody of the present invention is a member of the ICAM family and has five immunoglobulin-like C-type domains (NPLs 1 to 3). The amino acid sequence of human ICAM3, for example, is described in GenBank Accession No. NM_{—}002162. The amino acid sequence of human ICAM3 is shown in SEQ ID NO: 2, and the base sequence of a DNA encoding the amino acid sequence is shown in SEQ ID NO: 1. ICAM3 may be derived not only from human but also from other mammals (for example, rat, mouse, dog, cat, cattle, horse, sheep, pig, goat, rabbit, monkey). When the antibody of the present invention is used for treatment or diagnosis of human, human ICAM3 is targeted. In addition, ICAM3 may exist in a form having a naturally mutated amino acid, besides one having a typical amino acid sequence. Thus, ICAM3 targeted by the antibody of the present invention also includes natural mutants of amino acids. ICAM3 derived from mammals other than human and mutants of ICAM3 may differ from the human ICAM3 in amino acid sequence by one or more amino acids. Normally, such ICAM3 or the mutant is a polypeptide having a homology of 70% or more with the amino acid sequence of the above human ICAM3, preferably a polypeptide having a homology of 80% or more, further preferably a polypeptide having a homology of 90% or more, and more preferably a polypeptide having a homology of 95% or more.

In the present invention, when used as an antigen for producing the antibody of the present invention, “ICAM3” may be variants having one or more amino acids altered, besides ones having a natural amino acid sequence. An example of the aforementioned variant having one or more amino acids altered in the sequence is a polypeptide having a homology of 70% or more with the above amino acid sequence, preferably a polypeptide having a homology of 80% or more, further preferably a polypeptide having a homology of 90% or more, and more preferably a polypeptide having a homology of 95% or more. Furthermore, the variant may be a partial peptide of these ICAM3. In a case where an antibody drug or a diagnostic agent targeting human is to be developed, a human ICAM3 protein is preferable.

[Anti-ICAM3 Antibody]

The anti-ICAM3 antibody of the present invention may be a polyclonal antibody or a monoclonal antibody. The “polyclonal antibody” is an antibody preparation including different antibodies against different epitopes. Meanwhile, the “monoclonal antibody” means an antibody (including an antibody fragment) obtained from a substantially homogeneous antibody population. In contrast to the polyclonal antibody, the monoclonal antibody recognizes a single determinant on an antigen. The anti-ICAM3 of the present invention is preferably a monoclonal antibody. The origin, type, shape, and the like of the anti-ICAM3 antibody of the present invention are not particularly limited, as long as the anti-ICAM3 antibody binds to an ICAM3 protein. Specifically, it is possible to use antibodies such as an antibody derived from a non-human animal (for example, mouse antibody, rat antibody, camel antibody), a human antibody derived from human, a chimeric antibody, and a humanized antibody.

The anti-ICAM3 antibody of the present invention is preferably an anti-human ICAM3 antibody. The anti-human ICAM3 antibody may be an antibody specifically binding to human ICAM3, or may be an antibody binding to ICAM3 derived from other animals (for example, mouse ICAM3) besides human ICAM3.

The anti-ICAM3 antibody of the present invention may be obtained as a polyclonal antibody or a monoclonal antibody using known means. The anti-ICAM3 antibody of the present invention is particularly preferably a monoclonal antibody derived from a mammal. Examples of the monoclonal antibody derived from a mammal include ones produced by a hybridoma, ones produced by a host transformed with an expression vector containing an antibody gene by a genetic engineering technique, and the like.

The anti-ICAM3 antibody of the present invention may be modified with various molecules such as polyethylene glycol (PEG). In addition, as will be described later, the anti-ICAM3 antibody may be modified with a chemotherapy agent having a cytotoxic activity, a radioactive chemical substance, or the like.

The anti-ICAM3 antibody of the present invention is preferably an antibody having one or more activities described in (1) to (6) below.

(1) Cytotoxic Activity

In the treatment of cell proliferative diseases such as cancers, an antibody desirably retains its effector activity. Specifically, a preferable antibody in the present invention has both a binding affinity for ICAM3 and an effector function. The effector function of an antibody includes an antibody-dependent cell-mediated cytotoxicity (ADCC) activity and a complement-dependent cytotoxicity (CDC) activity. In the present invention, an antibody for treatment particularly preferably has an ADCC activity as the effector function. In a case where the antibody of the present invention is used for treatment, the antibody is preferably an antibody having a cytotoxic activity. Examples of the cytotoxic activity include an ADCC activity, a CDC activity, and the like. In the present invention, the “ADCC activity” means an activity to damage a target cell when an Fcγ receptor-bearing cell (an immune cell or the like) binds to an Fc portion of a specific antibody through the Fcγ receptor, the specific antibody having attached to a cell-surface antigen of the target cell. Meanwhile, the “CDC activity” means a cytotoxic activity by the complement system.

The present example demonstrated that the anti-ICAM3 antibody of the present invention even at a low concentration had an ADCC activity on cells expressing ICAM3 (Example 6). The anti-ICAM3 antibody of the present invention is preferably an antibody having an ADCC activity at 0.1 μg/ml on cells expressing ICAM3. Examples of the cells expressing ICAM3 include BaF3 cells expressing ICAM3 and various blood cancer cells (such as SKM-1 cell, U937 cell, KMS12BM cell). The ADCC activity of the anti-ICAM3 antibody used at 0.1 μg/ml is preferably, when evaluated by percentage of calcein released as described in the present example, 5% or more (10% or more, 15% or more, 20% or more, 25% or more, 30% or more, 35% or more, 40% or more, 45% or more, 50% or more, 55% or more) by percentage of calcein released.

Whether the anti-ICAM3 antibody has an ADCC activity or a CDC activity can be determined by known methods (for example, Current protocols in Immunology, Chapter 7. Immunologic studies in humans, Editor, John E, Coligan et al., John Wiley & Sons, Inc., (1993), and the like). Specifically, first, effector cells, complement solution, and target cells are prepared.

i) Preparation of Effector Cells

Effector cells can be prepared as follows. The spleen is extracted from a CBA/N mouse or the like, and the spleen cells are separated in an RPMI1640 medium (manufactured by Invitrogen Corporation). After washing with the same medium containing 10% fetal bovine serum (FBS, manufactured by HyClone Laboratories, Inc.), the cell concentration is adjusted to 5×10⁶/ml.

ii) Preparation of Complement Solution

A complement solution can be prepared from Baby Rabbit Complement (manufactured by Cedarlane Laboratories Limited) which is diluted 10-fold with a 10% FBS-containing medium (manufactured by Invitrogen Corporation).

iii) Preparation of Target Cells

Target cells can be prepared as follows. Cells expressing an ICAM3 protein are cultured together with 0.2 mCi of ⁵¹Cr-sodium chromate (manufactured by GE Healthcare Bio-Sciences Ltd.) in a 10% FBS-containing DMEM medium at 37° C. for one hour so that the target cells can be radioactively labeled. As the cells expressing an ICAM3 protein, it is possible to use cells transformed using a gene encoding the ICAM3 protein, various blood cancer cells (such as SKM-1 cell, U937 cell, KMS12BM cells), and the like. After the radioactive labeling, the cells are washed with a 10% FBS-containing RPMI1640 medium three times, and the cell concentration is adjusted to 2×10⁵/ml.

The ADCC activity or the CDC activity can be measured by methods described below. In a case where the ADCC activity is measured, the target cells and the anti-ICAM3 antibody are added each by 50 μl to a 96-well U-bottomed plate (manufactured by Becton Dickinson and Company), and are reacted on ice for 15 minutes. Then, 100 μl of the effector cells were added thereto, and cultured in a carbon dioxide gas incubator for 4 hours. The final concentration of the antibody is adjusted to 0 or 10 μg/ml. After the culturing, 100 μl of the supernatant is collected, and the radioactivity is measured with a gamma counter (COBRAII AUTO-GAMMA, MODEL D5005, manufactured by Packard Instrument Company). Using the obtained values, the cytotoxic activity (%) can be calculated according to the equation: (A−C)/(B−C)×100. A represents the radioactivity (cpm) of each sample, B represents the radioactivity (cpm) of a sample to which 1% NP-40 (manufactured by nacalai tesque, Inc.) has been added, and C represents the radioactivity (cpm) of a sample containing only the target cells.

Alternatively, instead of the method using radioactive labeling (⁵¹Cr-sodium chromate), the ADCC activity can be evaluated by measuring the fluorescent intensity (percentage of specific calcein released) using a fluorescent dye such as calcein AM (Wako Pure Chemical Industries, Ltd., 349-07201) as described in the present example. When this approach is employed, the cytotoxic activity (%) can be calculated, using the obtained values, according to the equation: (A−C)/(B−C)×100. A is a fluorescent value in each sample, B is an average fluorescent value of the cells lysed and released into a medium with Nonidet P-40 having a final concentration of 1%, and C is an average fluorescent value when only the medium was added.

On the other hand, in a case where the CDC activity is measured, the target cells and the anti-ICAM3 antibody are added each by 50 μl to a 96-well bottomed-plate (manufactured by Becton Dickinson and Company), and are reacted on ice for 15 minutes. Then, 100 μl of the complement solution was added thereto, and cultured in a carbon dioxide gas incubator for 4 hours. The final concentration of the antibody is adjusted to 0 or 3 μg/ml. After the culturing, 100 μl of the supernatant is collected, and the radioactivity is measured with a gamma counter. The cytotoxic activity can be calculated in the same manner as the measurement of the ADCC activity. The cytotoxic activity can be measured using a fluorescent dye similarly to the above.

Meanwhile, when the cytotoxic activity of an antibody conjugate is measured, the target cells and an anti-ICAM3 antibody conjugate are added each by 50 μl to a 96-well bottomed-plate (manufactured by Becton Dickinson and Company), and are reacted on ice for 15 minutes. The resultant is cultured in a carbon dioxide gas incubator for 1 to 4 hours. The final concentration of the antibody is adjusted to 0 or 3 μg/ml. After the culturing, 100 μl of the supernatant is collected, and the radioactivity is measured with a gamma counter. The cytotoxic activity can be calculated in the same manner as the measurement of the ADCC activity. The cytotoxic activity can be measured using a fluorescent dye similarly to the above.

(2) Cell-Proliferation-Suppressing Activity

A preferable embodiment of the anti-ICAM3 antibody of the present invention is an antibody having a cell-proliferation-suppressing activity. For example, in order to evaluate or measure the in vitro cell-proliferation-suppressing activity, ICAM3-expressing cells are seeded into a 96-well plate at an appropriate number per well. The anti-ICAM3 antibody is added thereto, and cultured approximately for several days to one week. Then, the number of viable cells can be counted using Cell Count Reagent SF (nacalai) in accordance with the attached instruction. As the negative control, PBS or the like may be added, or a control antibody having the same isotype may be added. The ICAM3-expressing cells are not particularly limited, and ICAM3-expressing BaF3 cells or various cancer cells (for example, blood cancer cell) can be used.

The present example demonstrated that the anti-ICAM3 antibody of the present invention even at a low concentration had a proliferation-suppressing activity on cells expressing ICAM3 (Example 7). The anti-ICAM3 antibody of the present invention is preferably an antibody having a proliferation-suppressing activity at 0.06 μg/ml (60 ng/ml) on cells expressing ICAM3. Particularly preferably, the antibody demonstrates a maximum proliferation-suppressing effect at 0.06 μg/ml (60 ng/ml) on cells expressing ICAM3. Examples of the cells expressing ICAM3 include various blood cancer cells (HL60 cell, SKM-1 cell, KMS12BM cell, IM9 cell, ARH77 cell, Kijk cell, and MC/CAR cell).

(3) In Vivo Anti-Tumor Activity

Another preferable embodiment of the anti-ICAM3 antibody of the present invention is an antibody having an anti-tumor activity in vivo. For example, in order to evaluate or measure the in vivo anti-tumor activity, ICAM3-expressing cancer cells are transplanted intravenously or subcutaneously into a non-human test animal. Then, from the day of the transplantation or the next day, a test antibody is intravenously or intraperitoneally administered every day or once every several days. By measuring the size of a tumor chronologically, the anti-tumor activity can be measured. As the negative control, PBS or the like may be administered, or a control antibody having the same isotype may be administered. If the sizes of tumors in the anti-ICAM3 antibody-administered group are smaller than the sizes of tumors in the negative control-administered group (although not particularly limited, for example, a ratio of tumor proliferation suppressed by 5-mg/kg administration is 20% or more, preferably 30% or more, and a ratio of tumor proliferation suppressed by 25-mg/kg administration is 30% or more, preferably 40% or more than that of the negative control-administered group), it can be determined that the antibody has an anti-tumor activity. The cancer cell is not particularly limited, but is preferably blood cancer cell (for example, leukemia cells such as SKM-1 cell). Moreover, the mouse to which the cancer cells are administered is not particularly limited, but is preferably a SCID mouse. More specifically, for example, it is possible to measure the anti-tumor activity of the antibody in vivo by the method described in Example in this application.

Further, as a preferable embodiment of the anti-ICAM3 antibody of the present invention, the anti-ICAM3 antibody alone preferably has an anti-tumor effect without requiring cross-linking with another antibody, or the like.

(4) Cross-Reaction

Another preferable embodiment of the anti-ICAM3 antibody of the present invention is an antibody which does not substantially bind to ICAM1 and/or ICAM5. Particularly preferably, the antibody binds to human ICAM3, but does not bind to human ICAM1 and/or human ICAM5. Whether or not an antibody binds to human ICAM1 and/or human ICAM5 can be checked by methods known to those skilled in the art. For example, it is possible to check by whether or not a test antibody binds to human ICAM1- or human ICAM5-expressing BaF3 cells. The sequences of human ICAM1 and human ICAM5 are known. For example, GenBank Accession No. X06990 and GenBank Accession No. U72671 can be referred to. An amino acid sequence of human ICAM1 is shown in SEQ ID NO: 4, and a DNA encoding the amino acid sequence is shown in SEQ ID NO: 3. Moreover, an amino acid sequence of human ICAM5 is shown in SEQ ID NO: 6, and a DNA encoding the amino acid sequence is shown in SEQ ID NO: 5.

(5) Internalization Activity

Moreover, the anti-ICAM3 antibody of the present invention may have an internalization activity. In the present invention, the “antibody having an internalization activity” means an antibody which is transported into the inside of a cell (into, for example, the cytoplasm, vesicle, and other organelles) when binding to ICAM3.

Whether or not an antibody has an internalization activity can be checked by methods known to those skilled in the art. For example, it is possible to check by: a method for checking whether or not a labeled substance bound to an anti-ICAM3 antibody is incorporated into a cell expressing ICAM3 by bringing the antibody into contact with the cell; a method for checking whether or not cell death is induced in an ICAM3-expressing cell by bringing an anti-ICAM3 antibody having a cytotoxic substance bound thereto into contact with the cell expressing ICAM3; or other methods.

The antibody having an internalization activity can be used as a pharmaceutical composition such as an anti-cancer agent to be described later, for example, by binding the aforementioned cytotoxic substance thereto.

(6) Apoptosis-Inducing Activity

Additionally, the anti-ICAM3 antibody of the present invention may have an apoptosis-inducing activity. Whether or not an antibody has an apoptosis-inducing activity can be checked by methods known to those skilled in the art (for example, Japanese Unexamined Patent Application Publication No. Hei 9-295999 and the like). For example, it is possible to check by methods such as detecting apoptosis by the MTS assay or flow cytometry after culturing, in the presence of a test antibody, ICAM3-expressing cells into which an ICAM3 gene has been introduced or human leukocyte cells.

The nature, shape, and the like of the anti-ICAM3 antibody of the present invention are not particularly limited, as long as the antibody binds to an ICAM3 protein. The anti-ICAM3 antibody of the present invention may be such antibodies as (a) to (f) below, for example.

(a) Conjugated Antibody

A cytotoxic substance such as a chemotherapy agent, a toxic peptide or a radioactive chemical substance may be bound to the anti-ICAM3 antibody of the present invention. Such a modified antibody (hereinafter referred to as an antibody conjugate) can be obtained by chemically modifying an obtained antibody. Note that the method for modifying an antibody has already been established in this field.

Examples of the chemotherapy agent bound to the anti-ICAM3 antibody to function the cytotoxic activity include chemotherapy agents as follow: azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, phenylbutyrate, prednisone, procarbazine, paclitaxel, pentostatin, semustine, streptozocin, tamoxifen, taxanes, taxol, testosterone propionate, thalidomide, thioguanine, thiotepa, teniposide, topotecan, uracil mustard, vinblastine, vinorelbine, and vincristine.

A preferable chemotherapy agent is a low-molecular-weight chemotherapy agent. The low-molecular-weight chemotherapy agent is less likely to interfere with the function of the antibody even after bound to the antibody. In the present invention, the low-molecular-weight chemotherapy agent has a molecular weight of normally 100 to 2000, preferably 200 to 1000. All of the chemotherapy agents exemplified herein are low-molecular-weight chemotherapy agents. These chemotherapy agents in the present invention include a prodrug which is converted into an active chemotherapy agent in vivo. The activation of such a prodrug may be through enzymatic conversion or nonenzymatic conversion.

In addition, the antibody may be modified with a toxic peptide. Examples of the toxic peptide include the followings: diphtheria toxin A chain (Langone J. J., et al., Methods in Enzymology, 93, 307-308, 1983), Pseudomonas exotoxin (Nature Medicine, 2, 350-353, 1996), ricin A chain (Fulton R. J., et al., J. Biol. Chem., 261, 5314-5319, 1986; Sivam G., et al., Cancer Res., 47, 3169-3173, 1987; Cumber A. J. et al., J. Immunol. Methods, 135, 15-24, 1990; Wawrzynczak E. J., et al., Cancer Res., 50, 7519-7562, 1990; Gheeite V., et al., J. Immunol. Methods, 142, 223-230, 1991); deglycosylated ricin A chain (Thorpe P. E., et al., Cancer Res., 47, 5924-5931, 1987); abrin A chain (Wawrzynczak E. J., et al., Br. J. Cancer, 66, 361-366, 1992; Wawrzynczak E. J., et al., Cancer Res., 50, 7519-7562, 1990; Sivam G., et al., Cancer Res., 47, 3169-3173, 1987; Thorpe P. E., et al., Cancer Res., 47, 5924-5931, 1987); gelonin (Sivam G., et al., Cancer Res., 47, 3169-3173, 1987; Cumber A. J. et al., J. Immunol. Methods, 135, 15-24, 1990; Wawrzynczak E. J., et al., Cancer Res., 50, 7519-7562, 1990; Bolognesi A., et al., Clin. exp. Immunol., 89, 341-346, 1992); pokeweed anti-viral protein from seeds (PAP-s) (Bolognesi A., et al., Clin. exp. Immunol., 89, 341-346, 1992); briodin (Bolognesi A., et al., Clin. exp. Immunol., 89, 341-346, 1992); saporin (Bolognesi A., et al., Clin. exp. Immunol., 89, 341-346, 1992); momordin (Cumber A. J., et al., J. Immunol. Methods, 135, 15-24, 1990; Wawrzynczak E. J., et al., Cancer Res., 50, 7519-7562, 1990; Bolognesi A., et al., Clin. exp. Immunol., 89, 341-346, 1992); momorcochin (Bolognesi A., et al., Clin. exp. Immunol., 89, 341-346, 1992); dianthin 32 (Bolognesi A., et al., Clin. exp. Immunol., 89, 341-346, 1992); dianthin 30 (Stirpe F., Barbieri L., FEBS letter 195, 1-8, 1986); Modeccin (Stirpe F., Barbieri L., FEBS letter 195, 1-8, 1986); viscumin (Stirpe F., Barbieri L., FEBS letter 195, 1-8, 1986); volkesin (Stirpe F., Barbieri L., FEBS letter 195, 1-8, 1986); dodecandrin (Stirpe F., Barbieri L., FEBS letter 195, 1-8, 1986); tritin (Stirpe F., Barbieri L., FEBS letter 195, 1-8, 1986); luffin (Stirpe F., Barbieri L., FEBS letter 195, 1-8, 1986); and trichokirin (Casellas P., et al., Eur. J. Biochem. 176, 581-588, 1988; Bolognesi A., et al., Clin. exp. Immunol., 89, 341-346, 1992).

In the present invention, a radioactive chemical substance refers to a chemical substance including a radioisotope. The radioisotope is not particularly limited, and any radioisotope may be used. For example, ³²P, ¹⁴C, ¹²⁵I, ³H, ¹³¹I, ¹⁸⁶Re, ¹⁸⁸Re, or the like can be used.

Moreover, in another embodiment, one of or two or more of the low-molecular-weight chemotherapy agent and the toxic peptide can be used in combination for modification of the antibody. In the binding of the anti-ICAM3 antibody and the low-molecular-weight chemotherapy agent, covalent bonding or non-covalent bonding can be utilized. Methods for producing antibodies to which these chemotherapy agents are bound are known.

The proteinaceous drug or toxin can be bound to the antibody according to a genetic engineering procedure. Specifically, for example, a recombinant vector can be constructed from an expression vector in which a DNA encoding the toxic peptide and a DNA encoding the anti-ICAM3 antibody are fused in-frame and incorporated. The vector is then introduced into an appropriate host cell to obtain transformed cells which are then cultured. The incorporated DNAs are expressed, and thus an anti-ICAM3 antibody to which the toxic peptide is bound can be obtained as a fusion protein. When a fusion protein with the antibody is to be obtained, generally, the proteinaceous drug or toxin is located at the C-terminal side of the antibody. A peptide linker may also be added between the antibody and the proteinaceous drug or toxin.

(b) Bispecific Antibody

The anti-ICAM3 antibody of the present invention may be a bispecific antibody. The bispecific antibody refers to an antibody having variable regions for recognizing different epitopes in the same antibody molecule. In the present invention, the bispecific antibody may have antigen binding sites for recognizing different epitopes on an ICAM3 molecule. Such a bispecific antibody enables two antibody molecules to bind to one ICAM3 molecule. As a result, a higher cytotoxic action can be expected.

Alternatively, the bispecific antibody may have one of the antigen binding sites recognizing ICAM3, and the other antigen binding site recognizing a cytotoxic substance. Specifically, the cytotoxic substance includes a chemotherapy agent, a toxic peptide, a radioactive chemical substance, or the like. Such a bispecific antibody binds to a cell expressing ICAM3, while trapping the cytotoxic substance. As a result, it is possible to make the cytotoxic substance directly act on ICAM3-expressing cells. In other words, the bispecific antibody recognizing a cytotoxic substance can specifically damage tumor cells and suppress proliferation of the tumor cells.

Further, in the present invention, it is also possible to use a bispecific antibody combined with an antigen binding site for recognizing an antigen other than ICAM3. For example, it is possible to produce a bispecific antibody combined with such an antigen binding site as to recognize an antigen which is different from ICAM3, and which is specifically expressed on the cell surface of a cancer cell targeted similarly to ICAM3.

The method for producing the bispecific antibody is known. For example, a bispecific antibody can be produced by combining two types of antibody which recognize different antigens. Each of the antibodies to be combined may be a ½ molecule having a heavy chain and a light chain, or may be a ¼ molecule consisting only of a heavy chain. Alternatively, a bispecific antibody-producing fusion cell can be produced by fusing hybridomas that produce different monoclonal antibodies from each other. Further, a bispecific antibody can be produced by a genetic engineering technique.

Known means can be used to measure the antigen binding activity of the antibody (Antibodies A Laboratory Manual. Ed Harlow, David Lane, Cold Spring Harbor Laboratory, 1988). For example, ELISA (enzyme-linked immunosorbent assay), EIA (enzyme immunoassay), RIA (radioimmunoassay), fluorescence immunoassay, or the like can be used.

(c) Sugar Chain-Modified Antibody

The anti-ICAM3 antibody of the present invention may be an antibody having a modified sugar chain. It is known that modifying a sugar chain of an antibody can enhance the cytotoxic activity of the antibody. Known examples as the antibody having a modified sugar chain include an antibody having a modified sugar chain (International Publication No. WO99/54342 and the like), an antibody deficient in fucose that is to be added to a sugar chain (International Publication Nos. WO00/61739, WO02/31140, and the like), an antibody having a sugar chain with bisecting GlcNAc (International Publication No. WO02/79255, and the like), and the like.

(d) Gene Recombinant Anti-ICAM3 Antibody

In a case where the antibody is to be administered to human, the antibody may be a gene recombinant antibody that is artificially modified so as to reduce the immunogenicity to human, or other purposes. Examples of the gene recombinant antibody include a chimeric antibody, a humanized antibody, and the like. These modified antibodies can be produced by known methods.

i) Chimeric Antibody

A chimeric antibody refers to an antibody in which variable regions and constant regions derived from different origins are linked to each other. For example, an antibody composed of variable regions of a heavy chain and a light chain of a mouse antibody and constant regions of a heavy chain and a light chain of a human antibody is a mouse-human-heterochimeric antibody. A recombinant vector which expresses a chimeric antibody can be produced from an expression vector in which DNAs encoding variable regions of a mouse antibody are ligated to DNAs encoding constant regions of a human antibody and incorporated. It is possible to obtain the chimeric antibody which is produced during culturing of recombinant cells transformed by the vector to thereby express the incorporated DNAs.

As the constant regions of the chimeric antibody, normally, those of a human antibody are used. For example, Cγ1, Cγ2, Cγ3, Cγ4, Cμ, Cδ, Cα1, Cα2, and Cε can be used as the constant region of the heavy chain. Moreover, Cκ and Cλ can be used as the constant region of the light chain. The amino acid sequence of these constant regions and the base sequences encoding these amino acid sequences are known. To improve the stability of the antibody itself or the stability of producing the antibody, one or more amino acids in the constant regions of the human antibody may be substituted, deleted, added and/or inserted.

ii) Humanized Antibody

Generally, a chimeric antibody includes variable regions of an antibody derived from an animal other than human and constant regions derived from a human antibody, while a humanized antibody includes complementarity determining regions (CDRs) of an antibody derived from an animal other than human, framework regions (FRs) derived from a human antibody, and constant regions derived from a human antibody. The humanized antibody is also called a reshaped human antibody. Specifically, there is known, for example, a humanized antibody obtained by grafting the CDRs of an antibody from an animal other than human, for example, mouse, into a human antibody. Since having a low immunogenicity in a human body, a humanized antibody is useful as an active ingredient of a therapeutic agent of the present invention.

A variable region of an antibody is normally composed of three CDRs flanked by four FRs. CDRs are regions substantially determining the binding specificity of an antibody. The amino acid sequence of a CDR is rich in diversity. Meanwhile, the amino acid sequence of an FR often shows a high homology even among antibodies having different binding specificities. For this reason, generally it is said that grafting CDRs enables transfer of the binding specificity of a certain antibody to another antibody.

General gene recombination techniques to obtain a humanized antibody are also known. Specifically, as a method for grafting a CDR of a mouse antibody to a human FR, for example, overlap extension PCR is known. In overlap extension PCR, a primer to which the base sequence encoding a CDR of a mouse antibody to be grafted is added is used as a primer for synthesizing an FR of a human antibody. The primer is prepared for each of four FRs. Generally, in grafting a mouse CDR to a human FR, it is said that selecting a human FR having a high homology with a mouse FR is advantageous to retain the function of the CDR. In other words, generally, it is preferable to use a human FR whose amino acid sequence has a high homology with the amino acid sequence of an FR adjacent to a mouse CDR to be grafted.

Moreover, base sequences to be ligated are designed to be connected to each other in-frame. Human FRs are individually synthesized by primers, respectively. As a result, a product is obtained in which DNAs encoding mouse CDRs are added to the FRs. In the product, the base sequences encoding mouse CDRs are designed to overlap each other. Subsequently, overlapped CDR portions anneal to each other for the complementary strand synthesis reaction. By this reaction, the human FRs are ligated to each other with the sequences of the mouse CDRs in between.

A full-length gene of the variable region in which three CDRs and the four FRs are ligated finally is amplified with primers which anneal to the 5′ end and the 3′ end, and to which an appropriate restriction enzyme recognition sequence is added. The DNA obtained as described above and a DNA encoding a human antibody constant region are inserted into an expression vector in such a manner as to be fused in-frame. Thereby, an expression vector for humanized antibody can be created. This vector is introduced into a host to establish recombinant cells. Then, the recombinant cells are cultured and the DNAs encoding the humanized antibody are expressed. Thus, the humanized antibody is produced in a culture of the cultured cells (see European Patent Application Publication No. 239400 and International Publication No. WO96/02576).

By qualitatively or quantitatively measuring and evaluating the binding activity of the humanized antibody produced as described above to an antigen, it is possible to suitably select such human antibody FRs that enable CDRs to form a favorable antigen binding site when the FRs are ligated with the CDRs in between. As necessary, amino acid residues of FRs may be substituted so that CDRs of the humanized antibody can form an appropriate antigen binding site. For example, it is possible to introduce a mutation into the amino acid sequence of an FR by applying the PCR used in grafting the mouse CDRs to the human FRs. Specifically, a partial base sequence mutation can be introduced into a primer annealing to an FR. The base sequence mutation is introduced in an FR synthesized by such a primer. By measuring and evaluating the binding activity of the mutant antibody with the substituted amino acid to an antigen by the above-described method, it is possible to select a mutated FR sequence having a desired characteristic (Sato, K. et al., Cancer Res, 1993, 53, 851-856).

(e) Polyvalent Antibody

As long as binding to an ICAM3 protein, the antibody of the present invention includes not only a bivalent antibody represented by an IgG (such as IgG1, IgG2, IgG4), but also a monovalent antibody or a polyvalent antibody represented by IgM. The polyvalent antibody of the present invention includes a polyvalent antibody having antigen binding sites that are all the same, or a polyvalent antibody having antigen binding sites that are partially or all different from each other.

(f) Low-Molecular-Weight Antibody

The antibody of the present invention is not limited to a full-length molecule of the antibody, and may be a low-molecular-weight antibody or a modified product thereof, as long as binding to an ICAM3 protein.

The low-molecular-weight antibody includes an antibody fragment missing a part of a whole antibody (for example, whole IgG, or the like). As long as having a binding ability to an ICAM3 antigen, an antibody molecule may be partially deleted. The antibody fragment in the present invention preferably includes any or both of a heavy chain variable region (VH) and a light chain variable region (VL). Moreover, the antibody fragment in the present invention preferably includes a CDR. The number of CDRs the antibody fragment of the present invention includes is not particularly limited, but the antibody fragment of the present invention preferably includes at least six: heavy chain CDR1, CDR2, CDR3, and light chain CDR1, CDR2, CDR3.

The amino acid sequence of VH or VL may include substitution, deletion, addition and/or insertion. Further, as long as the binding ability to an ICAM3 antigen is retained, any of VH and VL or part of both may be deleted. Moreover, the variable region may be chimeric or humanized. Specific examples of the antibody fragment include Fab, Fab′, F (ab′)2, Fv, and the like. Moreover, specific examples of the low-molecular-weight antibody include Fab, Fab′, F(ab′)2, Fv, scFv (single chain Fv), diabody, sc(Fv)2 (single chain (Fv)2), scFv-Fc, and the like. In the present invention, a preferable low-molecular-weight antibody is a diabody or sc(Fv)2. The low-molecular-weight antibody of the present invention also includes multimers (for example, dimer, trimer, tetramer, polymer) of these antibodies.

A fragment of the antibody can be obtained by treating the antibody with an enzyme to produce antibody fragments. The digestive enzyme cleaves the antibody fragment at a specific position, providing the antibody fragment with a specific structure. As the enzyme for producing an antibody fragment, for example, papain, pepsin, plasmin, or the like is known. By papain digestion, F(ab)2 or Fab is produced. By pepsin digestion, F(ab′)2 or Fab′ is produced. Alternatively, these antibody fragments can be expressed in an appropriate host cell after a gene encoding the antibody fragments is constructed and introduced into an expression vector (see, for example, Co, M. S. et al., J. Immunol. (1994) 152, 2968-2976, Better, M. & Horwitz, A. H. Methods in Enzymology (1989) 178, 476-496, Plueckthun, A. & Skerra, A. Methods in Enzymology (1989) 178, 497-515, Lamoyi, E., Methods in Enzymology (1986) 121, 652-663, Rousseaux, J. et al., Methods in Enzymology (1986) 121, 663-669, Bird, R. E. et al., TIBTECH (1991) 9, 132-137).

Any part of the antibody can be deleted from the enzymatically-obtained antibody fragment by using a genetic engineering technique. The low-molecular-weight antibody in the present invention may be an antibody fragment having any region deleted, as long as having the binding affinity to ICAM3.

i) Diabody

A diabody refers to a bivalent antibody fragment constructed by gene fusion (Holliger P et al., Proc. Natl. Acad. Sci. USA 90: 6444-6448 (1993), European Patent Application Publication No. 404,097, International Publication No. WO93/11161, and the like). A diabody is a dimer formed from two polypeptide chains. Normally, in each of the polypeptide chains forming a dimer, a heavy chain variable region and a light chain variable region are connected to each other on the same chain by a linker. The linker in a diabody is generally so short that the heavy chain variable region and light chain variable region cannot be connected to each other. Specifically, the amino acid residue forming the linker is, for example, approximately 5 residues long. Hence, the heavy chain variable region and the light chain variable region encoded on the same polypeptide chain cannot form a single-chain variable region fragment, but form a dimer together with a different single-chain variable region fragment. As a result, a diabody has two antigen binding sites.

ii) ScFv

An scFv obtained by linking together a heavy chain variable region and a light chain variable region of an antibody. In an scFv, the heavy chain variable region and the light chain variable region are linked by a linker, preferably a peptide linker (Huston, J. S. et al., Proc. Natl. Acad. Sci. U.S.A, 1988, 85, 5879-5883). The heavy chain variable region and the light chain variable region in an scFv may be derived from any antibody described in this description. The peptide linker linking the variable regions is not particularly limited. For example, any single-chain peptide made of approximately 3 to 25 residues can be used as the linker. Specifically, for example, peptide linkers to be described below or the like can be used.

The variable regions of both chains can be linked, for example, by PCR. To link the variable regions by PCR, among a DNA sequence encoding a heavy chain or a heavy chain variable region of an antibody, and a DNA sequence encoding a light chain or a light chain variable region of an antibody, a DNA encoding all or a desired part of an amino acid sequence is first used as a template.

The DNAs encoding the heavy chain and light chain variable regions are amplified by PCR using a pair of primers having sequences corresponding to the sequences of both ends of the DNA to be amplified. Next, a DNA encoding a peptide linker part is prepared. The DNA encoding the peptide linker can be synthesized using PCR, also. To the 5′ side of the primer used in this event, a base sequence that can be ligated to an amplified product of each variable region synthesized separately is added. Next, a PCR reaction is carried out using DNAs of [heavy chain variable region DNA]-[peptide linker DNA]-[light chain variable region DNA] and primers for assembly PCR.

The primers for assembly PCR are a combination of a primer annealing to the 5′ side of the [heavy chain variable region DNA] and a primer annealing to the 3′ side of the [light chain variable region DNA]. In other words, the primers for assembly PCR are a primer set that can amplify the DNAs encoding the full-length sequence of the scFv to be synthesized. Meanwhile, base sequences that can be ligated to the DNAs of the variable regions are added to the [peptide linker DNA]. As a result, these DNAs are ligated. Furthermore, the full-length scFv is finally produced as amplified products by the primers for assembly PCR. Once the DNAs encoding the scFv are produced, an expression vector containing these and recombinant cells transformed with the expression vector can be obtained in accordance with conventional processes. In addition, the scFv can be obtained by culturing the resulting recombinant cells and expressing the DNAs encoding the scFv.

iii) ScFv-Fc

An scFv-Fc is a low-molecular-weight antibody obtained by fusing an Fc region to an scFv (Cellular & Molecular Immunology 2006; 3: 439-443). The origin of the scFv used in an scFv-Fc is not particularly limited. For example, an scFv derived from IgM can be used. Moreover, the origin of the Fc is not particularly limited. For example, a human IgG (such as human IgG1) can be used. Thus, an example of a preferable embodiment of the scFv-Fc is an scFv-Fc in which a scFv fragment of an IgM antibody is linked to CH2 (for example, Cγ2) and CH3 (for example, Cγ3) of human IgG1 by a hinge region (Hγ) of human IgG1.

iv) Sc(Fv)2

An sc(Fv)2 is a single-stranded low-molecular-weight antibody obtained by linking two heavy chain variable regions (VH) and two light chain variable regions (VL) by a linker or the like (Hudson et al., J. Immunol. Methods 1999; 231: 177-189). An sc(Fv)2 can be produced by, for example, linking scFvs by a linker. To link four antibody variable regions, normally three linkers are needed.

In addition, a preferable antibody is characterized in that two VH and two VL are aligned in the order of VH, VL, VH, VL ([VH]-linker-[VL]-linker-[VH]-linker-[VL]) with the N-terminal side of a single-stranded polypeptide as the starting point.

The order of two VH and two VL is not particularly limited to the above configuration, and the regions may be aligned in any order. Examples of the order include the followings.

[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

As the linker linking variable regions of the antibody, it is possible to use any peptide linker that can be introduced by genetic engineering, linkers disclosed as a synthetic compound linker (see, for example, Protein Engineering, 9 (3), 299-305, 1996), and the like. The multiple linkers may be the same, or different linkers may be used. In the present invention, a peptide linker is preferable. The length of the peptide linker is not particularly limited, and can be selected as appropriate by those skilled in the art, depending on the purpose. Normally, the number of amino acid residues making up the peptide linker is 1 to 100 amino acids, preferably 3 to 50 amino acids, further preferably 5 to 30 amino acids, and particularly preferably 12 to 18 amino acids (for example, 15 amino acids).

The amino acid sequence of the peptide linker may be any sequence, as long as the binding action of the scFv is not inhibited. The amino acid sequence of the peptide linker can be selected as appropriate by those skilled in the art, depending on the purpose.

Hence, in the present invention, as a particularly preferable embodiment of the sc(Fv)2, for example, the following sc(Fv)2 can be cited:

[VH]-peptide linker (15 amino acids)-[VL]-peptide linker (15 amino acids)-[VH]-peptide linker (15 amino acids)-[VL].

Alternatively, the variable regions can be linked using a synthetic chemical linker (chemical crosslinking agent). Crosslinking agents normally used for crosslinking peptide compounds or the like can be used in the present invention. For example, chemical crosslinking agents as follow are known. These crosslinking agents are commercially available:
N-hydroxysuccinimide (NHS), disuccinimidyl suberate (DSS), bis(sulfosuccinimidyl)suberate (BS3), dithiobis(succinimidyl propionate) (DSP), dithiobis(sulfosuccinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), bis[2-(sulfosuccinimidooxycarbonyloxy)ethyl]sulfone (sulfo-BSOCOES), and the like.

Examples of the antibody which recognize and binds to ICAM3, and which is used in the present invention, include antibodies below:

(a) an antibody comprising heavy chain CDR1 having an amino acid sequence of SEQ ID NO: 15, heavy chain CDR2 having an amino acid sequence of SEQ ID NO: 16, heavy chain CDR3 having an amino acid sequence of SEQ ID NO: 17, light chain CDR1 having an amino acid sequence of SEQ ID NO: 22, light chain CDR2 having an amino acid sequence of SEQ ID NO: 23, and light chain CDR3 having an amino acid sequence of SEQ ID NO: 24;

(b) an antibody of (a) in which one or more amino acids are substituted, deleted, added and/or inserted, the antibody of (b) having an activity equivalent to that of the antibody of (a); and

(c) an antibody which recognizes a same epitope as an epitope recognized by the antibody of any one of (a) and (b).

In a case where the above-described antibodies comprise a constant region, the constant region to be used is not particularly limited, and any constant region may be used. An example of a preferable constant region used in the present invention is a human-derived constant region. For example, as a heavy chain constant region, it is possible to use a constant region derived from human IgG1, a constant region derived from human IgG2, a constant region derived from human IgG3, a constant region derived from human IgG4, or the like. Moreover, for example, as a light chain constant region, it is possible to use a constant region derived from human κ chain, a constant region derived from human λ chain, or the like. The amino acid sequence of the heavy chain constant region derived from human IgG1 is shown in SEQ ID NO: 26, and the base sequence of a DNA encoding the amino acid sequence is shown in SEQ ID NO: 25. Moreover, the amino acid sequence of the light chain constant region derived from human IgG1 is shown in SEQ ID NO: 28, and the base sequence of a DNA encoding the amino acid sequence is shown in SEQ ID NO: 27. The constant region used in the present invention may be a constant region having a natural sequence, or a variant of a constant region having a natural sequence in which one or more amino acids are modified.

In a case where the above-described antibody comprise an FR, the FR to be used is not particularly limited, and any FR may be used, as long as the binding activity to human ICAM3 is retained. Examples of the FR include FR1 to FR4 of a heavy chain variable region represented by amino acid sequences of SEQ ID NOs: 11 to 14, and FR1 to FR4 of a light chain variable region represented by amino acid sequences of SEQ ID NOs: 18 to 21. A preferable example of the FR used in the present invention is an FR derived from a human antibody. There is known a technique of substituting FRs while retaining the binding activity of an antibody against an antigen; accordingly, those skilled in the art can select an FR as appropriate. The FR used in the present invention may be an FR having a natural sequence, or an FR having a natural sequence in which one or more amino acids are modified.

An example of the antibody comprising heavy chain CDR1 having an amino acid sequence of SEQ ID NO: 15, heavy chain CDR2 having an amino acid sequence of SEQ ID NO: 16, heavy chain CDR3 having an amino acid sequence of SEQ ID NO: 17, light chain CDR1 having an amino acid sequence of SEQ ID NO: 22, light chain CDR2 having an amino acid sequence of SEQ ID NO: 23, and light chain CDR3 having an amino acid sequence of SEQ ID NO: 24 is an antibody comprising, as variable regions, a heavy chain variable region having an amino acid sequence of SEQ ID NO: 8 (IB23 heavy chain variable region) and a light chain variable region having an amino acid sequence of SEQ ID NO: 10 (IB23 light chain variable region). In a case where the antibody is a chimeric antibody, the antibody may comprise, as constant regions, the human-IgG heavy chain constant region described in SEQ ID NO: 26 and the human-IgG light chain constant region described in SEQ ID NO: 28.

In the present invention, having an activity equivalent to that of the antibody of the present invention refers to being equivalent in binding activity to ICAM3, cell-proliferation-suppressing activity, cell death-inducing activity, and cytotoxic activity (such as ADCC activity) on cells expressing ICAM3, and/or anti-tumor activity. In the present invention, to be equivalent in an activity, the activity does not necessary have to be identical. For example, such activities only need to be 50% or more, preferably 70% or more, and further preferably 90% or more, of the activities of the above-described antibodies. The upper limit of the activities is not particularly limited, and examples thereof include 1000% or less, 500% or less, 300% or less, 150% or less, 100% or less, and the like.

The antibody of the present invention in which one or more amino acids are substituted, deleted, added and/or inserted is also within the scope of the present invention. Such an antibody may be artificially produced, or naturally occur. An example of the method for introducing a mutation into a polypeptide is site-directed mutagenesis (Hashimoto-Gotoh, T. et al. (1995) Gene 152, 271-275, Zoller, M J, and Smith, M. (1983) Methods Enzymol. 100, 468-500, Kramer, W. et al. (1984) Nucleic Acids Res. 12, 9441-9456, Kramer W, and Fritz H J (1987) Methods. Enzymol. 154, 350-367, Kunkel, T A (1985) Proc Natl Acad Sci USA. 82, 488-492, Kunkel (1988) Methods Enzymol. 85, 2763-2766) or the like. This is one of methods well known to those skilled in the art to prepare a polypeptide functionally equivalent to a certain polypeptide. Those skilled in the art could prepare an antibody functionally equivalent to the antibody of the present invention by introducing a mutation into the antibody as appropriate using such a method. Meanwhile, such a mutation in an amino acid may naturally occur. In this manner, the antibody of the present invention also includes an antibody comprising an amino acid sequence of the antibody of the present invention in which one or more amino acids are mutated, the antibody being functionally equivalent to the antibody.

The number of amino acids mutated in such a mutant is normally 50 amino acids or less, preferably 30 amino acids or less, and further preferably 10 amino acids or less (for example, 5 amino acids or less).

The properties of an amino acid side chain of an amino acid residue to be mutated are desirably conserved in another amino acid thus mutated. For example, based on the properties of the amino acid side chain, categories as follow have been established:

hydrophobic amino acids (A, I, L, M, F, P, W, Y, V); hydrophilic amino acids (R, D, N, C, E, Q, G, H, K, S, T); amino acids having an aliphatic side chain (G, A, V, L, I, P); amino acids having a hydroxyl group-containing side chain (S, T, Y); amino acids having a sulfur atom-containing side chain (C, M); amino acids having a carboxylic acid- and amide-containing side chain (D, N, E, Q); amino acids having a base-containing side chain (R, K, H); and amino acids having an aromatic group-containing side chain (H, F, Y, W) (all of the amino acids are represented by single-letter codes in parentheses).

It has already been known that a polypeptide having an amino acid sequence which is modified from a certain amino acid sequence by deletion and/or addition of one or more amino acid residues and/or substitution with another amino acid retains the biological activity of the original polypeptide (Mark, D. F. et al., Proc. Natl. Acad. Sci. USA (1984) 81, 5662-5666, Zoller, M. J. and Smith, M., Nucleic Acids Research (1982) 10, 6487-6500, Wang, A. et al., Science 224, 1431-1433, Dalbadie-McFarland, G. et al., Proc. Natl. Acad. Sci. USA (1982) 79, 6409-6413). Specifically, generally, it is said that when an amino acid in the amino acid sequence of a certain polypeptide is substituted with an amino acid within the same category group, the activity of the polypeptide is highly likely to be retained. In the present invention, a substitution between amino acids within the same amino acid group described above refers to a conservative substitution.

The present invention also provides a peptide comprising a heavy chain or a light chain of an antibody comprising a CDR identified in the present invention, or a variable region of these chains. A preferable peptide is a peptide comprising the heavy chain of the antibody of the present invention having the amino acid sequences of SEQ ID NOs: 15 to 17, or a variable region thereof, and particularly preferably is a peptide comprising the amino acid sequence of SEQ ID NO: 8. Another preferable peptide is a peptide comprising the light chain of the antibody of the present invention having the amino acid sequences of SEQ ID NOs: 22 to 24, or a variable region thereof, and particularly preferably, a peptide comprising the amino acid sequence of SEQ ID NO: 10. By linking these peptides with, for example, a linker or the like, a functional antibody can be produced.

Moreover, the present invention also provides an antibody which binds to a same epitope as an epitope the above-described antibody binds to.

Whether or not a test antibody shares an epitope with a certain antibody can be checked by competition for the same epitope between the two. The competition between antibodies is detected by a cross-blocking assay or the like. For example, competitive ELISA assay is preferably a cross-blocking assay.

Specifically, in the cross-blocking assay, after preincubation of microtiter plate wells coated with an ICAM3 protein in the presence or absence of a candidate competing antibody, the anti-ICAM3 antibody of the present invention is added thereto. The amount of the anti-ICAM3 antibody of the present invention bound to the ICAM3 protein in the wells correlates indirectly with the binding ability of the candidate competing antibody (test antibody) that competes in binding to the same epitope. In other words, if the affinity of the test antibody to the same epitope increases, the amount of the anti-ICAM3 antibody of the present invention bound to the wells coated with the ICAM3 protein is decreased; meanwhile, the amount of the test antibody bound to the wells coated with the ICAM3 protein is increased.

The amount of an antibody bound to wells can be easily measured by labeling the antibody in advance. For example, a biotin-labeled antibody can be measured by using an avidin-peroxidase conjugate and an appropriate substrate. A cross-blocking assay using an enzyme label such as peroxidase particularly refers to a competitive ELISA assay. An antibody can be labeled with other labeled substances that can be detected or measured. Specifically, radiolabels, fluorescent labels, or the like are known.

Further, when a test antibody has a constant region derived from a species different from the anti-ICAM3 antibody of the present invention, an antibody bound to wells can be measured with a certain labeled antibody that recognizes the constant region. Alternatively, even when an antibody is derived from the same species but a different class, an antibody bound to wells can be measured with an antibody that can distinguish classes.

If a candidate antibody can block binding of the anti-ICAM3 antibody at least 20%, preferably at least 30%, further preferably at least 50%, and more preferably at least 80% in comparison with the binding activity obtained in a control test conducted in the absence of the competing antibody, the candidate antibody is an antibody which binds to substantially the same epitope as that of the anti-ICAM3 antibody of the present invention, or which competes in binding to the same epitope.

The antibody of the present invention is capable of exerting high cell-proliferation-suppressing activity, cell death-inducing activity, cytotoxic activity (for example, ADCC activity), and/or anti-tumor activity, and is therefore useful as a drug, particularly an anti-cancer agent.

[Production of Anti-ICAM3 Antibody]

1. Production of Anti-ICAM3 Antibody by Monoclonal Antibody-Producing Hybridoma

A hybridoma producing a monoclonal antibody can be produced according to known techniques as follows. First, an ICAM3 protein or a partial peptide thereof to be described later is used as a sensitizing antigen to immunize an animal according to a normal immunization method. The obtained immune cells are fused with known parent cells by a normal cell fusion method, and hybridomas are obtained. Further, the hybridomas are screened for cells producing a target antibody by a normal screening method, and thereby a hybridoma producing an anti-ICAM3 antibody is selected. A desired anti-ICAM3 monoclonal antibody is obtained from the hybridoma thus selected. Specifically, the following procedure is performed.

(1) Preparation of ICAM3 Protein

First, the ICAM3 gene is expressed, and thereby an ICAM3 protein used as the sensitizing antigen to obtain the antibody can be obtained. Specifically, the gene sequence encoding ICAM3 is inserted into a known expression vector and an appropriate host cell is transformed therewith. Then, from the host cell or culture supernatant, a target human ICAM3 protein is purified by a known method. A purified natural ICAM3 protein, or a fusion protein obtained by fusing a desired partial polypeptide of an ICAM3 protein with a different polypeptide, can also be used as the immunogen. To produce the fusion protein serving as the immunogen, for example, an antibody Fc fragment, a peptide tag, or the like can be used. A vector expressing the fusion protein can be produced by fusing genes encoding desired two or more kinds of polypeptide fragments in-frame, and inserting the fusion gene into an expression vector. The method for producing the fusion protein is described in Molecular Cloning, 2nd ed. (Sambrook, J et al., Molecular Cloning, 2nd ed., 9.47-9.58, Cold Spring Harbor Lab. press, 1989).

The ICAM3 protein purified in this manner can be used as the sensitizing antigen for use in the immunization of a mammal. A partial peptide of ICAM3 can also be used as the sensitizing antigen. For example, peptides as follow can be used as the sensitizing antigen.

The region and the size of ICAM3 used as the partial peptide are not limited. The number of amino acids making up the peptide serving as the sensitizing antigen is at least 3 or more, for example, 5 or more, or preferably 6 or more. More specifically, a peptide made of 8 to 50, preferably 10 to 30, residues can be used as the sensitizing antigen.

(2) Immunization with ICAM3 Protein

A mammal is immunized with the ICAM3 protein or the partial peptide as the sensitizing antigen. The mammal to be immunized is not particularly limited. However, in order to obtain a monoclonal antibody by the cell fusion method, it is preferable to select an immunized animal by taking the compatibility with the parent cells used for the cell fusion into consideration. Generally, rodent animals are preferable as the immunized animal. Specifically, mouse, rat, hamster, or rabbit can be used as the immunized animal. Besides, monkey and the like can also be used as the immunized animal.

The above animals can be immunized with the sensitizing antigen according to known methods. For example, as a general method, a mammal can be immunized by intraperitoneal or subcutaneous injection of the sensitizing antigen. Specifically, the sensitizing antigen is administered to the mammal several times every 4 to 21 days. The sensitizing antigen is diluted to an appropriate dilution with PBS (Phosphate-Buffered Saline), physiological saline, or the like for use in the immunization. Further, the sensitizing antigen may be coadministered with an adjuvant. For example, the sensitizing antigen may be prepared by mixing with Freund's complete adjuvant, followed by emulsification. Moreover, an appropriate carrier can be used in the immunization with the sensitizing antigen. Particularly, in a case where a partial peptide of a low molecular weight is used as the sensitizing antigen, the sensitizing antigen peptide is desirably attached to a carrier protein such as albumin or keyhole limpet hemocyanin for the immunization.

(3) DNA Immunization

The monoclonal antibody can also be obtained by DNA immunization. DNA immunization is a method for providing immune stimulation, including: administering to an animal to be immunized a vector DNA constructed in such a manner that a gene encoding an antigen protein can be expressed in the immunized animal; and expressing the immunizing antigen in the body of the immunized animal. In comparison with general immunization methods of administering a protein antigen, advantages as follow can be expected from the DNA immunization.
It is possible to provide immune stimulation while retaining the structure of a membrane protein such as ICAM3.
No purification of immunizing antigen is necessary.

To obtain a monoclonal antibody of the present invention by the DNA immunization, first, a DNA expressing the ICAM3 protein is administered to an animal to be immunized. The DNA encoding ICAM3 can be synthesized by known methods such as PCR. The obtained DNA is inserted into an appropriate expression vector, and administered to the animal to be immunized. As the expression vector, for example, commercially-available expression vectors such as pcDNA3.1 can be used. As the method for administering the vector into the body, generally-used methods can be employed. For example, the DNA immunization can be carried out by injecting gold particles having an expression vector adhered thereto into cells with a gene gun.

(4) Production of Hybridoma

When a desired increase in an antibody amount in a serum is observed after a mammal is immunized as described above, immune cells are collected from the mammal and subjected to cell fusion. As preferable immune cells, particularly spleen cells can be used.

As the cells fused with the immune cells, mammalian myeloma cells are used. The myeloma cells preferably include an appropriate selection marker for screening. The selection marker refers to such a trait that cells can (or cannot) survive under a specific culture condition. As the selection marker, hypoxanthine-guanine-phosphoribosyl transferase deficiency (hereinafter abbreviated as “HGPRT deficiency”), thymidine kinase deficiency (hereinafter abbreviated as “TK deficiency”), or the like is known. HGPRT- and TK-deficient cells are sensitive to hypoxanthine-aminopterin-thymidine (hereinafter abbreviated as “HAT sensitive”). HAT sensitive cells cannot synthesize DNA in a HAT selection medium and die. Meanwhile, when fused with normal cells, the HAT sensitive cells can utilize the salvage path of the normal cells and continue DNA synthesis, accordingly proliferating even in a HAT selection medium.

HGPRT-deficient or TK-deficient cells can be selected with a medium containing 6-thioguanine, 8-azaguanine (hereinafter abbreviated as “8AG”), or 5′-bromodeoxyuridine. Normal cells incorporate these pyrimidine analogues into the DNA and die. Meanwhile, cells deficient in these enzymes cannot incorporate these pyrimidine analogues and can survive in the selection medium. Besides, a selection marker called G418 resistance provides resistance to 2-deoxystreptamine antibiotics (gentamicin analogues) via a neomycin resistance gene. Various myeloma cells suitable for cell fusion are known. For example, myeloma cells as follow can be used for production of the monoclonal antibody in the present invention.

P3 (P3x63Ag8.653) (J. Immunol. (1979) 123, 1548-1550), P3x63Ag8U.1 (Current Topics in Microbiology and Immunology (1978) 81, 1-7), NS-1 (Kohler. G. and Milstein, C. Eur. J. Immunol. (1976) 6, 511-519), MPC-11 (Margulies. D. H. et al., Cell (1976) 8, 405-415), SP2/0 (Shulman, M. et al., Nature (1978) 276, 269-270), FO (de St. Groth, S. F. et al., J. Immunol. Methods (1980) 35, 1-21), S194 (Trowbridge, I. S. J. Exp. Med. (1978) 148, 313-323), R210 (Galfre, G. et al., Nature (1979) 277, 131-133), and the like.

Basically, cell fusion between the immune cells and the myeloma cells are carried out in accordance with a known method such as, for example, the method of Kohler and Milstein (Kohler. G. and Milstein, C., Methods Enzymol. (1981) 73, 3-46).

More specifically, the cell fusion can be carried out in a normal nutrient culture solution in the presence of a cell fusion promoter, for example. As the fusion promoter, for example, polyethylene glycol (PEG), Sendai virus (HVJ), and the like can be used. Further, an auxiliary substance such as dimethyl sulfoxide can be added, if desired, to increase the fusion efficiency.

Any ratio of immune cells to myeloma cells may be set for use. For example, immune cells are preferably 1 to 10 times as large as myeloma cells. As the culture solution used for the cell fusion, it is possible to use, for example, culture solutions normally used for this type of cell culturing such as an RPMI1640 culture solution, a MEM culture solution, and others that are suitable for proliferation of myeloma cell line. Further, a serum supplement such as fetal calf serum (FCS) can also be added to the culture solution.

In the cell fusion, predetermined amounts of immune cells and myeloma cells are mixed well in the culture solution, and mixed with a PEG solution preheated to approximately 37° C. Thus, target fusion cells (hybridomas) are formed. In the cell fusion method, for example, PEG having an average molecular weight of approximately 1000 to 6000 can be added normally at a concentration of 30 to 60% (w/v). Subsequently, a cell fusion agent and the like unfavorable for growth of the hybridomas are removed by repeating an operation of sequentially adding an appropriate culture solution noted above and removing the supernatant by centrifugation.

The hybridomas obtained in this manner can be selected by utilizing a selection culture solution corresponding to the selection marker of the myeloma used in the cell fusion. For example, cells having HGPRT- or TK-deficiency can be selected by culturing with a HAT culture solution (culture solution containing hypoxanthine, aminopterin and thymidine). Specifically, when HAT-sensitive myeloma cells are used in the cell fusion, cells successfully fused with normal cells can be selectively proliferated in a HAT culture solution. Culturing using the HAT culture solution is continued for a period enough to kill cells (non-fusion cells) but not the target hybridomas. Specifically, generally, the target hybridomas can be selected by culturing for several days to several weeks. Next, by conducting a normal limiting dilution procedure, the hybridoma producing the target antibody can be screened and monocloned.

The screening for the target antibody and monocloning can be suitably carried out by a screening method based on a known antigen-antibody reaction. For example, an antigen is bound to a carrier such as beads made of polystyrene or the like or a commercially-available 96-well microtiter plate, and is reacted with the culture supernatant of the hybridomas. Next, after the carrier is rinsed, an enzyme-labeled secondary antibody or the like is reacted therewith. If the target antibody reacting with the sensitizing antigen is contained in the culture supernatant, the secondary antibody binds to the carrier via the antibody. Finally, by detecting the secondary antibody binding to the carrier, whether or not the target antibody is present in the culture supernatant can be determined. The hybridoma producing a desired antibody having a binding ability to the antigen can be cloned by the limiting dilution procedure or the like. In this process, as the antigen, one used in the immunization or one that is substantially the same as the ICAM3 protein can be suitably used. For example, cell lines expressing ICAM3, soluble ICAM3, or the like can be used as the antigen.

To produce an antibody against human ICAM3, the method described in International Publication No. WO03/104453 can also be employed.

Moreover, instead of the method for obtaining the hybridoma by immunizing an animal other than human with an antigen, the target antibody can be obtained by sensitizing a human lymphocyte with an antigen.

Specifically, first, human lymphocytes are sensitized with an ICAM3 protein in vitro. Next, the immunosensitized lymphocytes are fused with an appropriate fusion partner. As the fusion partner, for example, it is possible to use myeloma cells which are derived from human and which are capable of dividing indefinitely (see Japanese Examined Patent Application Publication No. Hei 1-59878).

Further, the anti-ICAM3 human antibody can be obtained by administering an ICAM3 protein, as an antigen, to a transgenic animal having all the repertoires of the human antibody gene, or by immunizing such an animal with a DNA constructed in such a manner that ICAM3 is expressed in the animal. Antibody-producing cells of the immunized animal can be immortalized by a treatment such as cell fusion with an appropriate fusion partner or infection with Epstein-Barr virus. A human antibody against the ICAM3 protein can be isolated from the immortalized cells thus obtained (see International Publication Nos. WO94/25585, WO93/12227, WO92/03918, WO94/02602). Further, by cloning the immortalized cells, it is possible to clone cells producing an antibody having target reaction specificity. When a transgenic animal is used as the immunized animal, the immune system of the animal recognizes human ICAM3 as a foreign substance. In this manner, a human antibody against human ICAM3 can be easily obtained.

(5) Acquisition of Monoclonal Antibody from Hybridoma

The monoclonal antibody-producing hybridoma produced as described above can be subcultured in a normal culture solution. Moreover, the hybridoma can be preserved in liquid nitrogen for an extended period.

The target monoclonal antibody can be obtained from culture supernatant of the hybridoma cultured by a normal method. Alternatively, the monoclonal antibody can be obtained in the form of ascitic fluid by administering the hybridoma to a mammal compatible therewith and allowing the hybridoma to proliferate. The former method is suitable for obtaining a high purity antibody.

2. Production of Anti-ICAM3 Antibody by Genetic Engineering Technique

(1) Cloning of Antibody Gene

Using an antibody gene cloned from an antibody-producing cell, the antibody can be produced by employing a genetic engineering technique. The cloned antibody gene can be expressed as an antibody by incorporating the gene into an appropriate vector which is introduced into a host. The method for isolating the antibody gene, introducing it into the vector, and transforming the host cell has already been established (see, for example, Vandamme, A. M. et al., Eur. J. Biochem. (1990) 192, 767-775).

For example, a cDNA encoding a variable region of the anti-ICAM3 antibody can be obtained from cells of a hybridoma that produces the anti-ICAM3 antibody. For this, normally, first, total RNA is extracted from the hybridoma. As the method for extracting mRNA from the cell, for example, the following methods can be employed.
Guanidine ultracentrifugation method (Chirgwin, J. M. et al., Biochemistry (1979) 18, 5294-5299)
AGPC method (Chomczynski, P. et al., Anal. Biochem. (1987) 162, 156-159)

The extracted mRNA can be purified using mRNA Purification Kit (manufactured by GE Healthcare Bio-Sciences Ltd.) or the like. Alternatively, kits for extracting total mRNA directly from a cell, such as QuickPrep mRNA Purification Kit (manufactured by GE Healthcare Bio-Sciences Ltd.), are also commercially available. Using such a kit, total mRNA can be obtained from the hybridoma. The cDNA encoding the variable region of the antibody can be synthesized from the obtained mRNA using a reverse transcriptase. In this event, any sequence of 15-30 bases selected from a sequence common to antibody genes can be used as a primer. The cDNA can be synthesized with AMV Reverse Transcriptase First-strand cDNA Synthesis Kit (manufactured by Seikagaku Corporation) or the like. Moreover, to synthesize and amplify the cDNA, it is possible to use 5′-Ampli FINDER RACE Kit (manufactured by Clontech Laboratories, Inc.) and the 5′-RACE method utilizing PCR (Frohman, M. A. et al., Proc. Natl. Acad. Sci. USA (1988) 85, 8998-9002, Belyaysky, A. et al., Nucleic Acids Res. (1989) 17, 2919-2932). Further, in such a process of synthesizing the cDNA, an appropriate restriction enzyme site described below can be introduced to both ends of the cDNA.

A target cDNA fragment from the obtained PCR product is purified, and then ligated to a vector DNA. Recombinant vectors produced in this manner are introduced into Escherichia coli or the like. After colonies are selected, a desired recombinant vector can be prepared from colony-forming Escherichia coli. Then, the base sequence of the cDNA can be verified by a known method, for example, dideoxynucleotide chain termination method, or the like.

Furthermore, to obtain the gene encoding the variable region of the antibody, a cDNA library can also be used. First, a cDNA is synthesized using the mRNA extracted from the antibody-producing cells as a template to obtain a cDNA library. To synthesize the cDNA library, it is convenient to use a commercially-available kit. In practice, the amount of mRNA obtained only from a few cells is quite small, which results in a low yield if the mRNA is purified directly therefrom. Hence, normally, the mRNA is purified after a carrier RNA clearly not containing the antibody gene is added. Alternatively, if a certain amount of RNA can be extracted from the antibody-producing cells, efficient extraction is possible without addition of the carrier RNA. For example, the addition of the carrier RNA may not be necessary when the RNA is extracted from 10 or more, or or more, preferably 50 or more antibody-producing cells.

The antibody gene is amplified by PCR using the obtained cDNA library as a template. Primers for amplifying an antibody gene by PCR are known. For example, it is possible to design primers for amplification of a human antibody gene based on the disclosures of the article (J. Mol. Biol. (1991) 222, 581-597) and the like. The base sequences of these primers are different for each subclass of immunoglobulin. Hence, when a cDNA library of an unknown subclass is used as a template, PCR is carried out selecting a primer with all possibilities taken into consideration.

Specifically, for example, when a gene encoding human IgG is to be obtained, primers capable of amplifying genes encoding γ1 to γ5 as the heavy chain and κ and λ chains as the light chain can be used. To amplify an IgG variable region gene, generally a primer annealing to a part corresponding to a hinge region is used as the primer at the 3′ side. Meanwhile, primers corresponding to the respective subclasses can be used as the primer at the 5′ side.

The PCR products produced by gene-amplification primers for each subclass of heavy chain and light chain can be used as libraries independently to each other. Using the libraries thus synthesized, an immunoglobulin composed of a combination of heavy and light chains can be reshaped. The target antibody can be screened for based on the binding activity of the reshaped immunoglobulin to ICAM3.

(2) Introduction of Antibody Gene into Host Cell

To produce the anti-ICAM3 antibody, the cloned antibody gene is incorporated into an expression vector so that the gene can be expressed under the control of an expression regulatory region. The expression regulatory region for expressing the antibody includes, for example, an enhancer and a promoter. Next, a recombinant cell expressing the DNA encoding the anti-ICAM3 antibody can be obtained by transforming an appropriate host cell with this expression vector.

For the expression of the antibody gene, it is possible to incorporate DNAs encoding the heavy chain and the light chain of the antibody into different expression vectors. By co-transforming (co-transfecting) a single host cell with the vectors incorporating the heavy chain and the light chain, an antibody molecule comprising the heavy chain and the light chain can be expressed. Alternatively, the host cell may be transformed by incorporating the DNAs encoding the heavy chain and the light chain into a single expression vector (see International Publication No. WO94/11523).

Many combinations of host and expression vector are known which produce an antibody by introducing isolated antibody genes into an appropriate host. Any of these expression systems are applicable to the present invention. When a eukaryotic cell is used as the host, it is possible to use an animal cell, a plant cell, or a fungal cell. Specifically, examples of the animal cell that can be used in the present invention include mammalian cells such as CHO, COS, myeloma, BHK (baby hamster kidney), Hela, Vero, HEK293, Ba/F3, HL-60, Jurkat, and SK-HEP1; amphibian cells such as Xenopus laevis oocytes; and insect cells such as sf9, sf21, and Tn5.

As for the plant cell, an antibody gene expression system using cells derived from the genus Nicotiana such as Nicotiana tabacum is known. For the transformation of the plant cell, callus cultured cells can be used.

Further, as the fungal cell, the following cells can be used. For example, as for a yeast, examples thereof include yeasts belonging to the genus Saccharomyces such as Saccharomyces cerevisiae, and yeasts belonging to the genus Pichia such as methylotrophic yeast (Pichia pastoris). Moreover, as for a filamentous fungus, examples thereof include filamentous fungi belonging to the genus Aspergillus such as Aspergillus niger.

Alternatively, an antibody gene expression system using prokaryotic cells is also known. For example, when a bacterial cell is used, bacterial cells such as Escherichia coli and Bacillus subtilis can be used in the present invention.

When a mammalian cell is used, a construct containing a commonly-used, useful promoter, an antibody gene to be expressed, and a poly-A signal functionally ligated downstream at the 3′ side of the gene can be used for the expression. The construct may further contain an enhancer. An example of a promoter/enhancer is human cytomegalovirus immediate early promoter/enhancer.

Furthermore, a virus promoter/enhancer, a promoter/enhancer derived from mammalian cells such as human elongation factor 1α (HEF1α), or the like can be used for the antibody expression. Specific example of a virus capable of utilizing such a promoter/enhancer include retroviruses, polyomaviruses, adenoviruses, simian virus (SV40), and the like.

When an SV40 promoter/enhancer is used, the method of Mulligan et al. (Nature (1979) 277, 108) can be employed. Moreover, an HEF1α promoter/enhancer can be used easily for the expression of the target gene by the method of Mizushima et al. (Nucleic Acids Res. (1990) 18, 5322).

When an animal cell is used to produce the antibody, a signal sequence of the heavy chain gene or the light chain gene of the antibody is desirably used as a signal sequence necessary for the secretion to the outside of the cell. Further, the signal sequence of a secretory protein such as IL-3 or IL-6 can also be used.

In a case of Escherichia coli, a construct in which a commonly-used, useful promoter, a signal sequence for the antibody secretion, and an antibody gene to be expressed are functionally ligated can be used for the expression of the gene. Examples of the promoter include a lacZ promoter and an araB promoter. When the lacZ promoter is used, the method of Ward et al. (Nature (1989) 341, 544-546; FASEB J. (1992) 6, 2422-2427) can be employed. Alternatively, the araB promoter can be used for the expression of the target gene by the method of Better et al. (Science (1988) 240, 1041-1043).

For production in the periplasm of Escherichia coli, the pelB signal sequence (Lei, S. P. et al., J. Bacteriol. (1987) 169, 4379) may be used as the signal sequence for the antibody secretion. After the antibody produced in the periplasm is separated, the antibody structure can be refolded using a protein denaturing agent such as urea or guanidine hydrochloride so that the antibody has a desired binding activity.

As a replication origin to be inserted into the expression vector, it is possible to use ones derived from SV40, polyomaviruses, adenoviruses, bovine papilloma virus (BPV), and the like. Further, to amplify gene copies in the host cell system, a selection marker can be inserted into the expression vector. Specifically, it is possible to use selection markers such as aminoglycoside phosphotransferase (APH) gene, thymidine kinase (TK) gene, Escherichia coli xanthine guanine phosphoribosyl transferase (Ecogpt) gene, and dihydrofolate reductase (dhfr) gene.

(3) Acquisition of Antibody from Host Cell

These expression vectors are introduced into host cells, and then the transformed host cells are cultured in vitro or in vivo to produce the target antibody. The host cells can be cultured by known methods. For example, as the culture solution, DMEM, MEM, RPMI1640, or IMDM can be used. A serum supplement such as fetal calf serum (FCS) can be used in combination.

The antibody expressed and produced as described above can be purified by employing known methods normally used in protein purification alone or in combination as appropriate. For example, the antibody can be separated and purified by appropriately selecting and combining an affinity column such as a protein A column, a chromatography column, and filter, as well as procedures such as ultrafiltration, salting out, and dialysis (Antibodies A Laboratory Manual. Ed Harlow, David Lane, Cold Spring Harbor Laboratory, 1988).

Thus, the present invention provides a gene encoding the antibody of the present invention. Moreover, the present invention provides a vector comprising the gene. Further, the present invention provides a host cell comprising the vector. Furthermore, the present invention provides a method for producing an antibody encoded by the gene, the method comprising the step of culturing the host cell.

3. Production of Antibody by Transgenic Animal

Besides the above-described host cell, a transgenic animal can also be used to produce a recombinant antibody. In other words, the target antibody can be obtained from an animal into which the gene encoding the target antibody is introduced. For example, the antibody gene can be constructed as a fusion gene by inserting the antibody gene in-frame within a gene encoding a protein that is produced specifically in milk. As the protein secreted into milk, for example, goat β-casein and the like can be used. A DNA fragment containing the fusion gene having the antibody gene inserted is injected into a goat embryo, and the injected embryo is introduced into a female goat. A transgenic goat (or progeny thereof) born from the embryo-receiving goat produces milk from which the desired antibody can be obtained as a fusion protein with the milk protein. Additionally, a hormone can be used as appropriate in a transgenic goat so as to increase the amount of milk containing the desired antibody that is produced from the transgenic goat (Ebert, K. M. et al., Bio/Technology (1994) 12, 699-702).

[Pharmaceutical Composition]

The anti-ICAM3 antibody is useful for treatment against a cancer expressing ICAM3, and so forth. Specifically, the present invention provides a pharmaceutical composition comprising the anti-ICAM3 antibody as an active ingredient. In a certain embodiment, the pharmaceutical composition of the present invention is a cell-proliferation depressant, particularly an anti-cancer agent. The cell-proliferation depressant and the anti-cancer agent of the present invention are preferably administered to a subject who has a cancer or a subject who may have a cancer.

The anti-ICAM3 antibody used in the pharmaceutical composition (for example, anti-cancer agent) of the present invention is not particularly limited. For example, any of the anti-ICAM3 antibodies described above can be used. In the present invention, the phrase “comprising the anti-ICAM3 antibody as an active ingredient” means comprising the anti-ICAM3 antibody as a primary active component, and does not limit the content ratio of the anti-ICAM3 antibody.

In a case where the disease targeted by the pharmaceutical composition of the present invention is a cancer, the targeted cancer is not particularly limited, but is preferably leukemia (for example, acute myeloid leukemia and the like), myeloma, and malignant lymphoma. The cancer may be any of primary cancer and metastatic cancer.

The pharmaceutical composition of the present invention can be administered to a patient by any of oral administration and parenteral administration. Parenteral administration is preferable. Specific examples of the administration method include injection administration, transnasal administration, transpulmonary administration, transdermal administration, and the like. Examples of the injection administration include intravenous injection, intramuscular injection, intraperitoneal injection, subcutaneous injection, and the like. By carrying out these administration methods, the pharmaceutical composition of the present invention can be systemically or locally administered. Moreover, the administration method can be selected as appropriate in accordance with the age and condition of the patient. As the dose, for example, the dose in a single administration can be selected from the range of 0.0001 mg to 1000 mg per kg body weight. Alternatively, for example, the dose can be selected from the range of 0.001 to 100000 mg per patient. However, the pharmaceutical composition of the present invention is not limited to these doses.

The pharmaceutical composition of the present invention can be formulated in accordance with a conventional process (for example, Remington's Pharmaceutical Science, latest edition, Mark Publishing Company, Easton, U.S.A), and may also comprise a pharmaceutically acceptable carrier or an additive in addition to the active ingredient. Examples of the carrier and additive include surfactants, excipients, coloring agents, flavoring agents, preservatives, stabilizers, buffers, suspensions, isotonic agents, binders, disintegrators, lubricants, fluidity improvers, taste masking agents, and the like. Without limitation thereto, other commonly-used carriers and additives can be further used as appropriate. Specific examples of the carrier include light anhydrous silicic acid, lactose, crystalline cellulose, mannitol, starch, carmellose calcium, carmellose sodium, hydroxypropyl cellulose, hydroxypropyl methylcellulose, polyvinyl acetal diethylaminoacetate, polyvinyl pyrrolidone, gelatin, medium chain fatty acid triglycerides, polyoxyethylene hydrogenated caster oil 60, white sugar, carboxymethyl cellulose, corn starch, inorganic salts, and the like.

When brought into contact with ICAM3-expressing cells, the anti-ICAM3 antibody of the present invention can induce cell death in the cells, damage, and/or suppress the cell proliferation. In the present invention, “contact” is carried out, for example, by adding the antibody to a culture solution of ICAM3-expressing cells cultured in vitro. Further, in the present invention, “contact” is carried out also by administering the antibody to a non-human animal into which ICAM3-expressing cells have been transplanted or an animal having a cancer cell endogenously expressing ICAM3. Such methods for using the anti-ICAM3 antibody of the present invention are also within the scope of the present invention. The anti-ICAM3 antibody to be used is not particularly limited. For example, any of the anti-ICAM3 antibodies described above can be used. The cell to which the anti-ICAM3 antibody binds is not particularly limited, as long as the cell expresses ICAM3. A preferable ICAM3-expressing cell in the present invention is a cancer cell. The anti-ICAM3 antibody of the present invention is more preferably used against leukemia, myeloma, or malignant lymphoma.

[Diagnostic Agent (Diagnosis Method)]

Furthermore, the present invention provides a diagnosis method for a cancer, characterized by comprising the step of:

detecting any one of an ICAM3 protein and a gene encoding the ICAM3 protein. The expression level of ICAM3 has been found significantly high in cancer cell lines, particularly leukemia (for example, acute myeloid leukemia and the like) and myeloma. Hence, ICAM3 is useful as a marker for specifically detecting a cancer.

One of specific examples of the diagnosis method of the present invention is a diagnosis method for a cancer, comprising the steps of:

preparing a sample isolated from a subject; and

detecting a level of any one of an ICAM3 protein and an ICAM3 gene expressed in the sample. The method of the present invention may further comprise a step of:

evaluating a possibility that the subject has a cancer on a basis of the level of any one of an ICAM3 protein and an ICAM3 gene expressed.

(1) Detection of ICAM3 Protein

One embodiment of the method of the present invention is to diagnose a cancer by detecting an ICAM3 protein in a sample. Preferably, the ICAM3 protein is detected by using an antibody for recognizing the ICAM3 protein.

In the present invention, detection is meant to include quantitative or qualitative detection. Examples of the qualitative detection include measurement only for whether or not the ICAM3 protein is present, measurement for whether or not the ICAM3 protein is present in a certain amount or larger, measurement to compare the amount of the ICAM3 protein with that of another sample (for example, a control sample or the like), and other similar measurements. Meanwhile, examples of the quantitative detection include measurement for the concentration of the ICAM3 protein, measurement for the amount of the ICAM3 protein, and other similar measurements.

In the present invention, a test sample is not particularly limited, as long as there is a possibility that the sample contains an ICAM3 protein. Specifically, a sample collected from a body of an organism such as a mammal is preferable. A further preferable sample is a sample collected from human. Specific examples of the test sample include blood, interstitial fluid, plasma, extravascular fluid, cerebrospinal fluid, synovial fluid, pleural fluid, serum, lymph, saliva, urine, tissue, and the like. A preferable sample is a sample obtained from a test sample such as a specimen of fixed tissue or cells collected from a body of an organism, or a culture solution of the cells.

The cancer to be diagnosed by the present invention is not particularly limited and may be any cancer. Specific examples thereof include leukemia (for example, acute myeloid leukemia and the like) and myeloma. In the present invention, any of primary lesions and metastatic lesions can be diagnosed.

In the present invention, if the protein is detected in a test sample, the diagnosis of the cancer is given using the level of the protein as an indicator. Specifically, if the amount of the ICAM3 protein detected in a test sample is larger than that of a negative control or healthy subjects, this indicates that the subject has a cancer or is likely to develop a cancer in the future. In other words, the present invention relates to a diagnosis method for a cancer, comprising the steps of:

detecting an ICAM3 expression level in a biological sample collected from a subject (step (1)); and

determining that the subject has a cancer if the ICAM3 expression level detected in the step (1) is higher than that of a control (step (2)).

In the present invention, the control refers to a sample serving as a reference for comparison, and includes negative controls and biological samples from healthy subjects. A negative control can be obtained by collecting biological samples from healthy subjects, and mixing together as necessary. The ICAM3 expression level of the control can be detected concurrently with the ICAM3 expression level in a biological sample from a subject. Alternatively, the ICAM3 expression level in biological samples from a large number of healthy subjects is detected in advance, and a standard expression level of the healthy subjects is statistically determined. The standard value thus determined can be used as a control value. Specifically, for example, mean value±2×standard deviation (S.D.), or mean value±3×standard deviation (S.D.), can be used as a standard value. Statistically, the mean value±2×standard deviation (S.D.) includes the values of 80% of the healthy subjects, and the mean value±3×standard deviation (S.D.) includes the values of 90%.

Alternatively, the ICAM3 expression level in the control can be set using an ROC curve. The ROC curve (receiver operating characteristic curve) is a graph showing the detection sensitivity on the vertical axis and the false-positive rate (i.e., “1-specificity”) on the horizontal axis. In the present invention, an ROC curve can be obtained by plotting changes in sensitivity and false-positive rate when the reference value for determining the ICAM3 expression level in a biological sample is varied continuously.

Note that the “reference value” for obtaining the ROC curve is a numerical value used temporarily for statistical analysis. Generally, the “reference value” for obtaining the ROC curve is continuously varied within a range that can cover all the reference values possibly selected. For example, the reference value can be varied between the minimum value and the maximum value of the measured values of ICAM3 in the population to be analyzed.

Based on the obtained ROC curve, a desired detection sensitivity and a standard value expected to be accurate can be selected. A standard value statistically set with an ROC curve or the like is also called a cut-off value. In the detection method for a cancer based on a cut-off value, the ICAM3 expression level detected in the step (1) is compared with the cut-off value in the step (2). Then, if the ICAM3 expression level detected in the step (1) is higher than the cut-off value, a cancer is detected in the subject.

In the present invention, the ICAM3 expression level can be determined by any method. Specifically, the ICAM3 expression level can be estimated by evaluating the amounts of the ICAM3 mRNA and the ICAM3 protein or the biological activity of the ICAM3 protein. The amounts of the ICAM3 mRNA and protein can be determined by the methods described in this description.

In the present invention, a particularly suitable subject is human. Note that when an animal other than human is a subject, the ICAM3 protein in the animal species is detected. The method for detecting the ICAM3 protein included in a test sample is not particularly limited, but detection using the anti-ICAM3 antibody by immunological methods as exemplified below is preferable.

Enzyme-linked immunosorbent assay (ELISA), radioimmunoassay (RIA), enzyme immunoassay (EIA), fluorescence immunoassay (FIA), luminescence immunoassay (LIA), immunoprecipitation (IP), turbidimetric immunoassay (TIA), western blotting (WB), immunohistochemistry (IHC), single radial immunodiffusion (SRID), dot blotting, and slot blotting.

Among these procedures, immunohistochemistry (IHC) includes the step of detecting an ICAM3 protein on a section of fixed tissue or cells obtained from a patient having a cancer, and is one of immunological assays preferable as the diagnosis method for a cancer. The above-described immunological methods such as immunohistochemistry (IHC) are methods known to those skilled in the art.

Since ICAM3 is a membrane protein with specifically enhanced expression in cancer cells, cancer cells (including cancer cells in cancer tissues) can be detected by the anti-ICAM3 antibody. Thus, cancer cells included in cells and tissue collected from a body are detected by the above immunohistological analysis.

In another preferable embodiment, cancer cells in a body can be detected with the anti-ICAM3 antibody. To trace the antibody administered to the body, it is possible to use an anti-ICAM3 antibody which is detectably labeled. To be more specific, this method includes the steps of: administering an anti-ICAM3 antibody labeled with a labeled substance, such as a radioisotope, to a subject; and detecting accumulation of the labeled substance. For example, using a fluorescent substance, a luminescent substance, or a radioisotope as the labeled substance, the behavior of an antibody labeled with these in the body is traced, and thereby cancer cells in the body can be detected. The antibody labeled with a fluorescent substance or a luminescent substance can be observed by using an endoscope or laparoscope. When a radioisotope is used as the labeled substance, the localization of the antibody can be imaged by tracing the radioactivity. In the present invention, the localization of the anti-ICAM3 antibody in the body indicates the presence of cancer cells.

As the radioisotope for labeling the antibody to detect a cancer in a body, positron emitting nuclides can be used. For example, the antibody can be labeled with positron emitting nuclides such as 18F, 55Co, 64Cu, 66Ga, 68Ga, 76Br, 89Zr, and 124I. For labeling of the anti-ICAM3 antibody with these positron emitting nuclides, a known method (Acta Oncol. 32, 825-830, 1993) can be employed.

After the anti-ICAM3 antibody labeled with positron emitting nuclides is administered to human or an animal, the radiation emitted from the radioactive nuclide is measured from the outside of the body with a PET (positron emission tomography device), and converted to an image by a computer tomography procedure. The PET is a device for obtaining data on the behavior and the like of a drug in the body non-invasively. With the PET, the emission intensity can be imaged quantitatively as a signal strength. The use of the PET as described above enables detection of an antigen molecule that is highly expressed in a specific cancer without collecting a sample from a patient. The anti-ICAM3 antibody may also be radiolabeled with a short-lived nuclide using a positron emitting nuclide such as 11C, 13N, 15O, 18F, and 45Ti besides the above-described nuclides.

Research and development have been progressed concerning: production of short-lived nuclides using the above nuclides with a medical cyclotron; manufacturing techniques of short-lived radiolabeled compounds; and the like. By these techniques, an anti-ICAM3 antibody can be labeled with various radioisotopes. The anti-ICAM3 antibody administered to a patient accumulates in primary and metastatic lesions by the specificity of the anti-ICAM3 antibody against the pathological tissue at each site. When the anti-ICAM3 antibody is labeled with a positron emitting nuclide, the presence of primary and metastatic lesions can be detected according to the localization of the radioactivity. For the diagnostic application, a gamma-particle or positron emission energy of 25-4000 keV as an activity value can be suitably used. In addition, by selecting an appropriate nuclide and further by administering the nuclide in a large amount, a therapeutic effect can also be expected. To obtain an anti-cancer action by radiation, a nuclide providing a gamma-particle or positron emission energy of 70-700 keV can be used.

(2) Detection of Polynucleotide Encoding ICAM3 Protein

In another embodiment of the method of the present invention, expression of a polynucleotide of ICAM3 is detected. In the present invention, the polynucleotide to be detected is not particularly limited, but an mRNA is preferable. In the present invention, detection is meant to include quantitative or qualitative detection. Examples of the qualitative detection include measurement only for whether or not the ICAM3 mRNA is present, measurement for whether or not the ICAM3 mRNA is present in a certain amount or larger, measurement to compare the amount of the ICAM3 mRNA with that of another sample (for example, a control sample or the like), and other similar measurements. Meanwhile, examples of quantitative detection include measurement for the concentration of the ICAM3 mRNA, measurement for the amount of the ICAM3 mRNA, and other similar measurements.

In the present invention, as the test sample, any sample that possibly contains the ICAM3 mRNA can be used. The sample is preferably a sample collected from a body of an organism such as a mammal, further preferably a sample collected from human. Specific examples of the test sample include blood, interstitial fluid, plasma, extravascular fluid, cerebrospinal fluid, synovial fluid, pleural fluid, serum, lymph, saliva, urine, tissue, and the like. The test sample of the present invention also includes a sample obtained from a test sample such as a specimen of fixed tissue or cells collected from a body of an organism, or a culture solution of the cells.

An in situ hybridization method is suitably employed when a sample used is obtained from a test sample such as a specimen of fixed tissue or cells collected from a body of an organism, or a culture solution of the cells. The in situ hybridization method has been developed as means for verifying the presence or distribution of a specific DNA or RNA in cells and tissues, and the degree of the expression. As the principle, the method utilizes the nature of a probe nucleic acid having a base sequence complementary to the sequence of a specific nucleic acid in a cell, the probe nucleic acid specifically forming a complex. If the probe is labeled in advance with radioisotope (RI), an antigenic substance (hapten), or the like, detecting the label makes a hybridized site distinguishable. For this reason, the in situ hybridization method is employed for detection of DNA, RNA, and the like in cells. As the label for the probe, a radioisotope can be suitably used. Other suitable examples of the label include fluorescent labels utilizing a hapten such as a non-radioactive substance biotin and digoxigenin, and the like. An example of a particularly suitable detection method is a detection method by fluorescence in situ hibridization, so-called FISH.

The cancer diagnosed by the present invention is not particularly limited. Specific examples thereof include leukemia and myeloma. In the present invention, any of primary lesions and metastatic lesions can be diagnosed.

In the present invention, any animal species expressing the ICAM3 gene can be a subject. A particularly suitable subject is human. Note that when an animal species other than human is a subject, the ICAM3 gene of the animal species is detected.

A specific embodiment of the detection method will be described below. First, a sample is prepared from a subject. Next, the ICAM3 mRNA included in the sample is detected. In the present invention, a cDNA synthesized from the mRNA can also be detected. In the present invention, when the ICAM3 mRNA or the cDNA encoding ICAM3 is detected in the test sample, it is concluded that there is a possibility of a cancer. For example, when the amount of the ICAM3 mRNA or cDNA encoding ICAM3 detected in the test sample is larger than that of a negative control or healthy subjects, this indicates that the subject has a cancer or is likely to develop a cancer in the future.

The method for detecting an mRNA is known. Specifically, for example, using a solidified sample selected from a gene chip, a cDNA array, and a membrane filter, nucleic acid hybridization, RT-PCR, real-time PCR, subtraction technique, differential display, differential hybridization, cross hybridization, and the like can be employed in the present invention.

The detection method of the present invention may be automated using various automatic detection devices. The automation enables inspection of a large number of samples in a short period of time.

[Kit for Diagnosing Cancer]

The present invention also provides a diagnostic agent or a kit for diagnosing a cancer, the diagnostic agent or the kit comprising a reagent for detecting an ICAM3 protein in a test sample. The diagnostic agent of the present invention comprises at least the anti-ICAM3 antibody.

A kit for diagnosing a cancer can be prepared by combining the reagent for diagnosing a cancer of the present invention with another component used for detection of ICAM3. In other words, the present invention relates to a kit for diagnosing a cancer which comprises an antibody binding to ICAM3 and a reagent for detecting binding between the antibody and the ICAM3, and which may further comprise a control sample of a biological sample including ICAM3. The kit of the present invention may further comprise an instruction for explaining measurement procedure.

### EXAMPLES

Hereinafter, the present invention will be more specifically described based on Examples and comparative example. However, the present invention is not to be limited to Examples below.

### Example 1

### Acquisition of Mouse Anti-ICAM3 Antibody

(1) Establishment of ICAM3-Expressing BaF3

An ICAM3 (GenBank No. NM_{—}002162) cDNA fragment (SEQ ID NO: 1) was introduced into an expression vector for animal cell. The expression vector thus prepared was introduced into Ba/F3 cells by electroporation, and an ICAM3-expressing BaF3 transfectant (ICAM3/BaF3) was established. Note that the base sequence was checked using BigDye Terminator Cycle Sequencing Kit (Applied Biosystems) and DNA Sequencer ABI PRISM 3700 DNA Sequencer (Applied Biosystems) in accordance with the attached instructions.

(2) Production of Mouse Anti-ICAM3 Antibody-Producing Hybridoma

i) Production of Hybridoma Using ICAM3/BaF3 Cell-Immunized Mice

For immunization, 4-week-old male MRL/lpr mice (Charles River Laboratories Japan, Inc.) were used. The ICAM3/BaF3 cells were suspended in 100 μl of PBS at a cell concentration of 1×10⁷ to 3×10⁷ cells. This was intravenously administered to the mice. After the weekly immunization four times, the mouse spleen cells and mouse myeloma cells P3X63Ag8U.1 (hereinafter P3U1, ATCC CRL-1597) were subjected to cell fusion using PEG1500 (Roche Diagnostics) in accordance with a conventional process. The fusion cells (i.e., hybridomas) were cultured in a HAT medium (RPMI1640+PS, 10% FCS, HAT (Sigma, H0262) with 5% BM condimed H1 (Roche: #1088947)).

ii) 1.2.2. Selection of Hybridoma Cells

Approximately one week after the cell fusion, primary screening was carried out based on cell agglutination-inducing activity. The cell agglutination-inducing activity was checked as follows. The ICAM3/BaF3 cells were seeded into a 96-well plate at 4×10³ cells/well in 25 μl, and 80 μl of the culture supernatant of each hybridoma was added thereto, followed by culturing at 37° C. overnight. Each well was observed with a microscope, and wells in which the cells agglutinated were visually determined. Hybridoma cells in the wells with cell agglutination were selected as positive clones.

Subsequently, secondary screening was carried out as follows. KMS-12-BM cells were reacted with the hybridoma culture supernatant, and the binding activity to the KMS-12-BM cells was analyzed by FACS. From the hybridomas, clones producing an antibody strongly binding to the KMS-12-BM cells were selected as positive clones.

The hybridoma cells in the selected wells were seeded again into a 96-well plate at 1 cell/well, followed by culturing for approximately 10 days. Then, the binding activities to the BaF3 cells and ICAM3/BaF3 were examined by FACS, and monoclones specifically reacting with ICAM3/BaF3 were selected. Thereby, hybridomas producing the anti-ICAM3 monoclonal antibody were established.

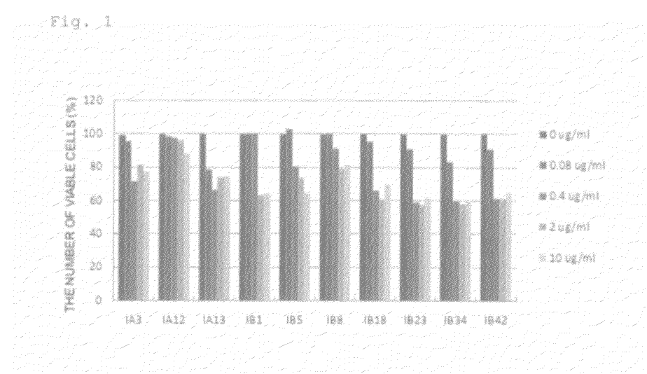

Among monoclonal antibodies obtained in this manner, a clone having the highest proliferation-suppressing activity was selected by the following procedure. First, the antibodies were purified from the hybridoma culture supernatant using Hi Trap Protein G HP 1 ml column (Amersham Biosciences #17-0404-01) in accordance with the attached instruction. Using the purified antibodies, the cell-proliferation-suppressing activity on KMS-12-BM cells was analyzed as follows. KMS-12-BM cells were seeded into a 96-well plate at 1×10⁴ cells/well. The purified antibodies were added to each well at 0 to 10 μg/ml, followed by culturing for 6 days. Then, the number of viable cells was counted using Cell Count Reagent SF (nacalai) in accordance with the attached instruction. Based on this measurement result, a clone IB23 having the highest cell-proliferation-suppressing activity was selected (FIG. 1).

### Example 2

### Comparison of Binding Activities of IB23 Antibody on Various Blood Cancer Lines

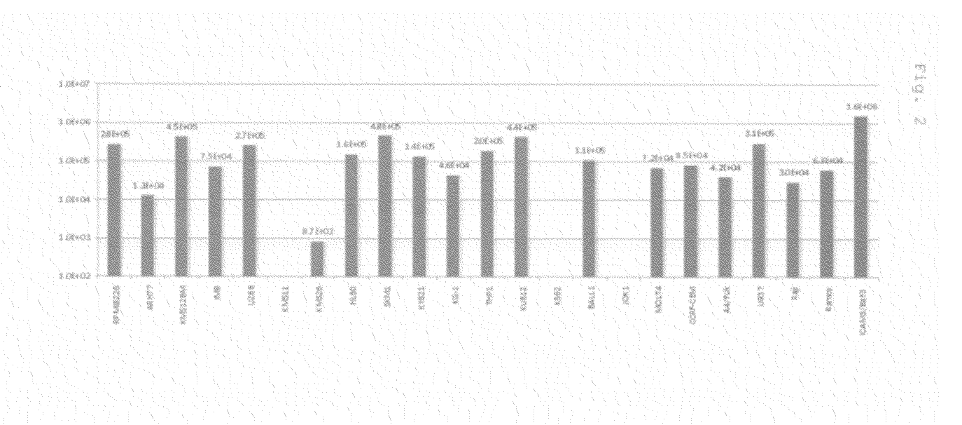

The number of cell surface ICAM3 antigens on various blood cancer lines was calculated based on a binding activity unit of the IB23 antibody. Table 1 shows a list of the blood cancer cell lines used in the measurement. The number of ICAM3 antigens (i.e., binding units of the IB23 antibody) on these cells was analyzed using QIFI KIT (manufactured by Dako, K0078). The various blood cancer cell lines were stained with a mouse anti-IB23 antibody (30 μg/ml), and the binding activity was measured by FACS analysis. Based on this binding activity (fluorescent intensity), the number of ICAM3 antigens was obtained by calculation in accordance with the attached instruction. FIG. 2 shows the result.

TABLE 1

| Call line | Cancer type | Origin |
|---|---|---|
| RPMI8226 | myeloma | Japan Health Sciences Foundation |
| ARH77 | myeloma | ATCC |
| KMS12BM | myeloma | Japan Health Sciences Foundation |
| IM9 | myeloma | ATCC |
| U266 | myeloma | ATCC |
| KMS11 | myeloma | Japan Health Sciences Foundation |
| KMS26 | myeloma | Japan Health Sciences Foundation |
| HL60 | AML | Japan Health Sciences Foundation |
| SKM1 | AML | Japan Health Sciences Foundation |
| KY821 | AML | Japan Health Sciences Foundation |
| KG-1 | AML | Japan Health Sciences Foundation |
| THP1 | AML | Japan Health Sciences Foundation |
| KU812 | CML | Japan Health Sciences Foundation |
| K562 | CML | Japan Health Sciences Foundation |
| BALL1 | B-ALL | Japan Health Sciences Foundation |
| JOK1 | B-CLL | Fujisaki Cell Center, |
| | | Hayashibara Biochemical Labs., |
| | | Inc. |
| MOLT4 | T-ALL | Japan Health Sciences Foundation |
| CCRF-CEM | T-ALL | Dainippon Pharmaceutical Co. |
| | | Ltd. |
| A4/Fuk | lymphoma | Japan Health Sciences Foundation |
| U937 | lymphoma | Japan Health Sciences Foundation |
| Raji | lymphoma | Japan Health Sciences Foundation |
| Ramos | lymphoma | Japan Health Sciences Foundation |
| ICAM3/BaF3 | Transfectant | Construted by ourselves |

### Example 3

### Analysis on Cross-Reactivity with ICAM Family

ICAM3 has a high homology with ICAM1 and ICAM5 among the ICAM family molecules. Hence, the cross-reactivity with these family molecules was analysed.

A human ICAM1 (GenBank No. X06990) cDNA fragment (SEQ ID NO: 3) was amplified by PCR using human lung marathon-ready cDNA (takara, 50629) as a template, and a human ICAM5 (GenBank No. U72671) cDNA fragment (SEQ ID NO: 5) was amplified by PCR using human brain marathon-ready cDNA (takara, 50598) as a template. The products were introduced into expression vectors for animal cell. The base sequences were checked using BigDye Terminator Cycle Sequencing Kit (Applied Biosystems) and DNA sequencer ABI PRISM 3700 DNA Sequencer (Applied Biosystems) in accordance with the attached instructions. Each of the produced expression vectors was introduced into Ba/F3 cells. Thus, an ICAM1-expressing BaF3 transfectant (ICAM1/BaF3) and ICAM5-expressing BaF3 transfectant (ICAM5/BaF3) were established.

Next, using these cell lines thus established, the cross-reactivity of IB23 with the ICAM family was analyzed. Each 10 μg/ml of the IB23 antibody, an anti-human ICAM1 antibody (R&D Systems, Inc., #BBA3), and an anti-human ICAM5 antibody (R&D Systems, Inc., #MAB1950) were reacted with ICAM1/BaF3 and ICAM5/BaF3. Then, after staining with an anti-mouse IgG-FITC antibody (Beckman Coulter #IM0819), the binding of each antibody was analyzed by FACS (Becton, Dickinson and Company).

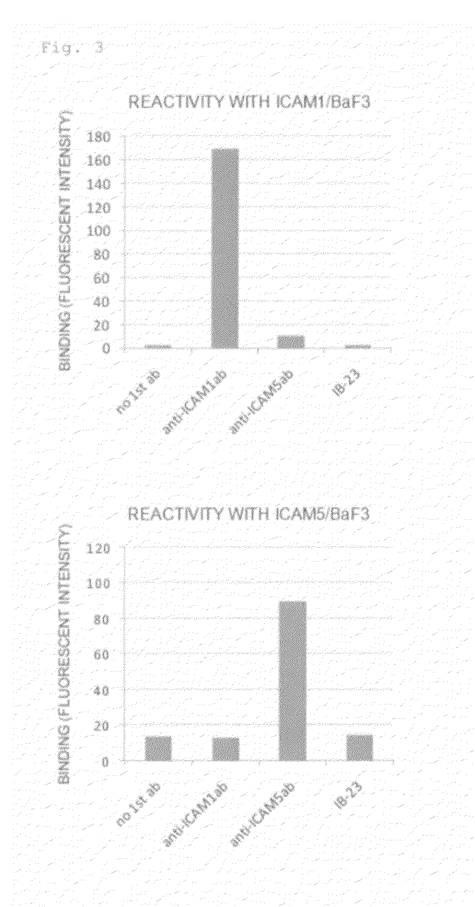

As a result, it was revealed that IB23 did not bind to ICAM1/BaF3 nor ICAM5/BaF3, but specifically reacted with ICAM3 (FIG. 3).

### Example 4

### Determination of Variable Regions of Mouse Anti-ICAM3 Antibody: IB23

Total RNA was extracted from the hybridoma cells using RNeasy Mini Kits (QIAGEN), and a cDNA was synthesized with SMART RACE cDNA Amplification Kit (BD Biosciences). Using the produced cDNA as a template, DNAs corresponding to variable regions of the antibody were amplified by PCR. The obtained genes of the variable regions of the antibody were inserted into a cloning vector. The base sequence of each DNA fragment was determined using BigDye Terminator Cycle Sequencing Kit (Applied Biosystems) and DNA sequencer ABI PRISM 3700 DNA Sequencer (Applied Biosystems) in accordance with the attached instructions. The determined base sequence of the heavy chain variable region of the mouse IB23 antibody is shown in SEQ ID NO: 7, and that of the light chain variable region is shown in SEQ ID NO: 9. CDRs and FRs were determined according to Kabat numbering.

### Example 5

### Production of IB23 Human Chimeric Antibody

A gene fragment obtained by linking the heavy chain variable region (SEQ ID NO: 7) of the mouse IB23 antibody to a heavy chain constant region (SEQ ID NO: 25) of human IgG1 was introduced into an expression vector for animal cell. Moreover, a gene fragment obtained by linking the light chain variable region (SEQ ID NO: 9) of the mouse IB23 antibody to a light chain constant region (SEQ ID NO: 27) of human IgG1 was introduced into an expression vector for animal cell. The antibody was expressed and purified by the method described in Reference Example 1 below.

### Example 6

### Production of Deglycosylated IB23 Human Chimeric Antibody

The followings are known from literatures. If asparagine at position 297 in the Fc region of human IgG1 is substituted with alanine and deglycosylated, this weakens binding of effector cells to an Fc receptor. Thereby, the ADCC activity by the effector cells is lost. For this reason, a deglycosylated human chimeric antibody was produced from the IB23 antibody.

According to the literature information (Cancer Res 2008, 68, 9832-9838), a heavy chain constant region (CH_N297A) (SEQ ID NO: 29) was produced by substituting alanine for asparagine at position 297 in the EU numbering in the human IgG1 Fc region. The heavy chain variable region (SEQ ID NO: 7) of the mouse IB23 antibody was linked to CH_N297A to thereby form a heavy chain, which was then introduced into an expression vector for animal cell.

Moreover, the light chain variable region (SEQ ID NO: 9) of the mouse IB23 antibody was linked to the light chain constant region (SEQ ID NO: 27) of human IgG1 to thereby form a light chain, which was then introduced into an expression vector for animal cell. The antibody was expressed and purified by the method described in Reference Example 1 below.

### Example 7

### Proliferation-Suppressing Activity of IB23

Antibody on Various Blood Cancer Lines

Using human blood cancer cell lines expressing ICAM3, the cell-proliferation-suppressing activity of the IB23 human chimeric IgG1 antibody was measured as described below.

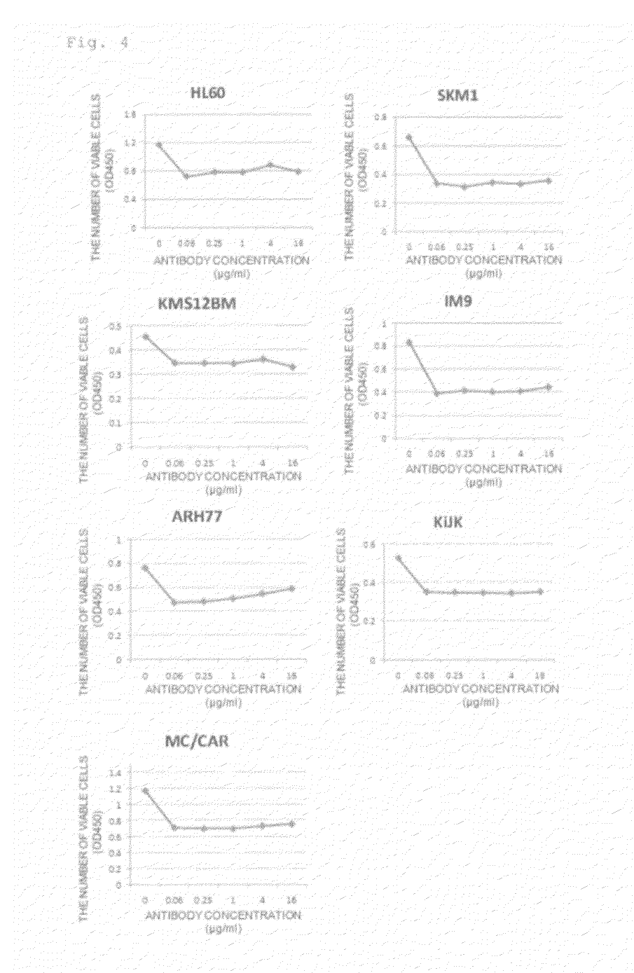

Various blood cancer cell lines were seeded into a 96-well plate at 3×10³ to 6×10³ cells/well to which the purified IB23 human chimeric antibody was added so that the final concentration was 0 to 16 μg/ml. After culturing for 4 days, the number of viable cells was counted using WST-8 (cell counting kit-8). The measurement was performed with n=3, and a mean value was calculated for each measurement result. As a result, as shown in FIG. 4, the IB23 human chimeric antibody exhibited a cell-proliferation-suppressing activity on the various blood cancer cell lines. It was revealed that the activity demonstrated a maximum proliferation-suppressing effect even at a concentration as low as 60 ng/ml. It can be seen from this result that the IB23 antibody alone is capable of suppressing cell proliferation quite considerably.

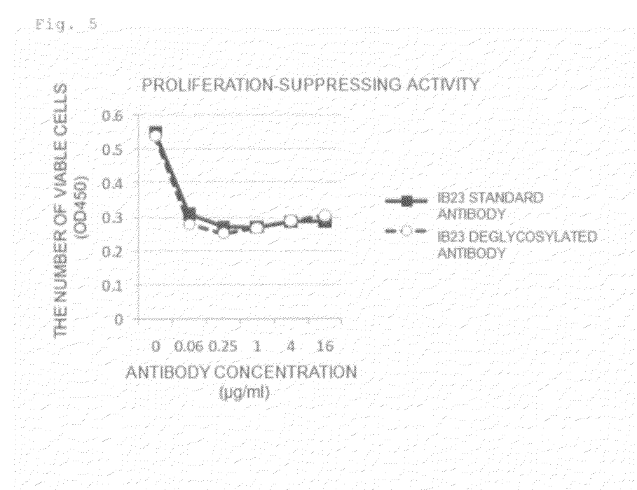

Next, it was examined whether or not a difference was observed between the IB23 human chimeric antibody and the deglycosylated IB23 human chimeric antibody in cell-proliferation-suppressing activity. SKM-1 cells were seeded into a 96-well plate at 3×10³ cells/well. To such plates, each of the IB23 human chimeric antibody (standard antibody) and the deglycosylated IB23 human chimeric antibody was separately added in the same manner as the above. The number of viable cells was counted using WST8 (cell counting kit-8), and the proliferation-suppressing activity was compared between the two antibodies. As a result, it was verified that the proliferation-suppressing activity of even the deglycosylated type did not differ from that of the standard IB23 antibody (FIG. 5).

### Example 8

### Analysis on ADCC Activity of IB23 Antibody

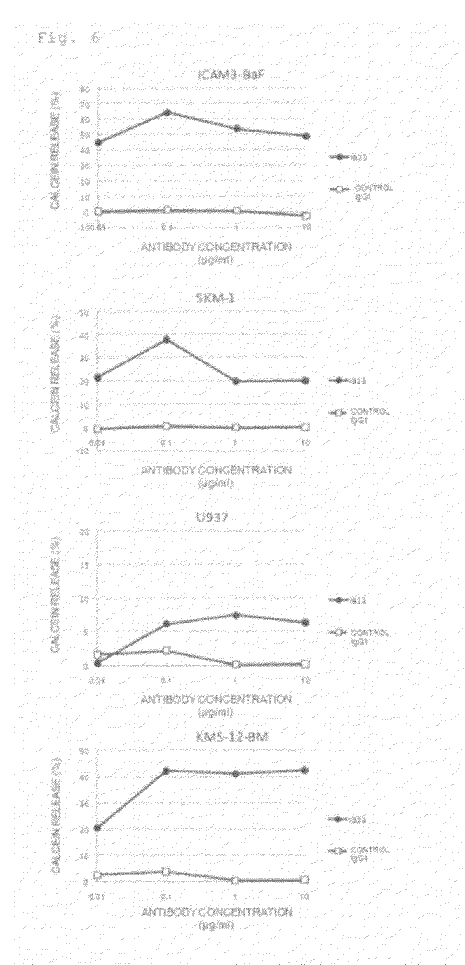

The ADCC activity of the IB23 standard antibody (human chimeric IgG1) on the blood cancer cell lines were analyzed. The ADCC activity was analyzed by an calcein release assay. Calcein AM (Wako Pure Chemical Industries, Ltd., 349-07201) was added to each of ICAM3/BaF3 cells, SKM-1 cells, U937 cells, and KMS-12-BM cells which had been respectively seeded into 96-well plates. By culturing for 90 minutes, calcein was incorporated into the cells. Then, the cells were washed with a culture solution and suspended in a fresh culture solution. Subsequently, the antibody was added thereto. Further, to each well, effector cells (recombinant cells of NK-92 [ATCC, CRL-2407] which were forced to express mouse Fc-gamma receptor 3 [NM_{—}010188]) were added in an amount approximately 5 times as large as the target cell. Each of the plates was allowed to stand in a 5% CO₂ incubator at 37° C. for 4 hours. After allowed to stand, the plate was centrifuged, and a certain amount of supernatant was collected from each well to measure the fluorescent intensity of calcein released into the medium by the cell injury (λex=490 nm, λem=515 nm). As a result, as shown in FIG. 6, the IB23 standard antibody induced a quite high ADCC activity not only on the ICAM3/BaF3 but also on the blood cancer cell lines. This revealed that the IB23 antibody alone was capable of not only suppressing cell proliferation but also inducing the ADCC activity.

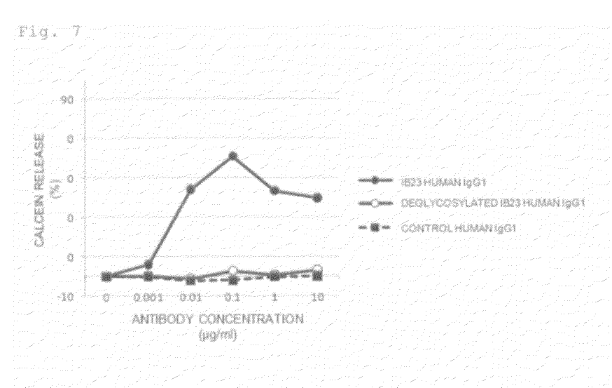

Next, the ADCC activity was compared between the IB23 standard antibody and the IB23 deglycosylated antibody and analyzed using ICAM3/BaF3 cells as a target cell. FIG. 7 shows the result. It was verified that a quite high ADCC activity was observed in the standard antibody, and that the deglycosylation of the antibody made the ADCC activity completely lost.

Note that the percentage of specific calcein released was calculated according to the equation: “percentage of specific calcein released (%)=(A−C)×100/(B−C).” Here, A is the fluorescent value in each well, B is an average fluorescent value of the cells lysed and released into the medium with Nonidet P-40 having a final concentration of 1%, and C is an average fluorescent value when only the medium was added.

### Example 9

### Analysis on In Vivo Anti-Tumor Activity of IB23 Antibody

A human leukemia cell line, SKM-1 cells (1×10⁷ cells), was prepared in RPMI1640 (SIGMA Cat. No. R8758) and subcutaneously transplanted into the abdomen of SCID mice (Icr-scid Jcl, female, 6 weeks old, CLEA Japan, Inc.) to which 0.2 mg of an anti-asialo GM1 antibody (manufactured by Wako Pure Chemical Industries, Ltd.) had been intraperitoneally administered on the day before. The engraftment of tumor was checked, and the mice were grouped into 5 on day 23 after the transplantation according to the tumor volume and body weight (one control group, four drug-administered group, each n=6).

On the day of grouping (day 23) and on day 30, PBS was intravenously administered to the control group at 10 mL/kg, while the IB23 standard antibody and the IB23 deglycosylated antibody were intravenously administered to the drug administered groups at doses of 5 mg/kg and 25 mg/kg, respectively. Then, the tumor volume was measured over time, and plotted on a graph.

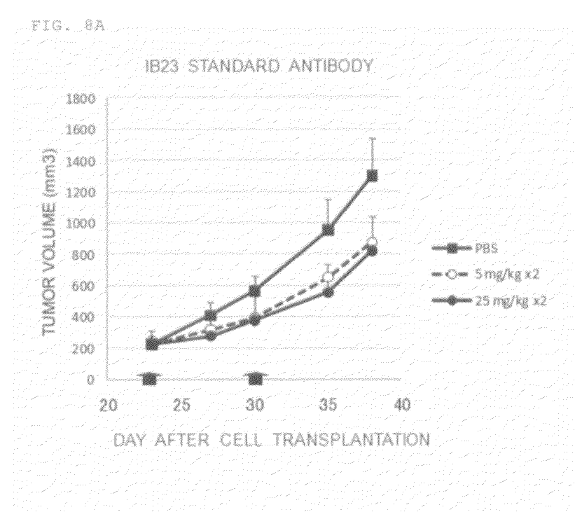

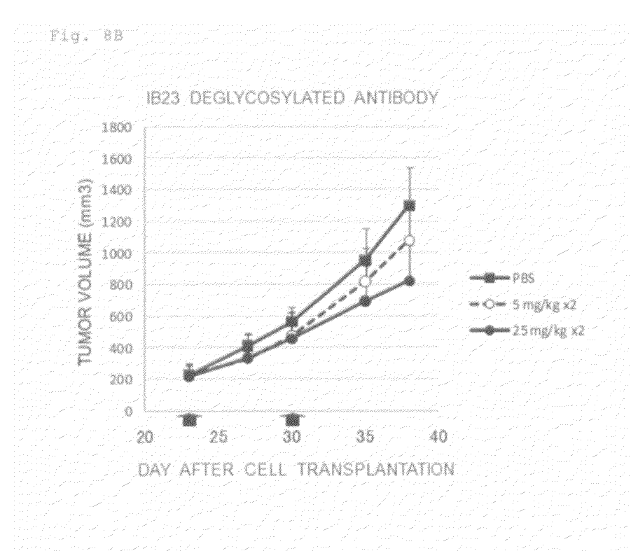

FIG. 8 shows the result. When the measurement was completed, the ratio of tumor proliferation suppressed by each dose of the IB23 standard antibody was 39% in the 5-mg/kg administered group and 44% in the 25-mg/kg administered group (FIG. 8A). Meanwhile, even by the administration of the IB23 deglycosylated antibody having no ADCC activity, the ratio of tumor proliferation suppressed was found to be 29% in the 5-mg/kg administered group and 43% in the 25-mg/kg administered group (FIG. 8B).

Since the tumor regression was observed by the IB23 deglycosylated antibody having no ADCC activity, it was verified that a sufficient anti-tumor action was satisfactorily exerted only by the proliferation-suppressing activity of the IB23 antibody. Moreover, since a superior drug action was observed in the standard antibody-administered groups, it was suggested that in addition to the proliferation-suppressing activity, the ADCC activity additively acted and enhanced the anti-tumor action of the IB23 antibody.

### Reference Example 1

### Production of Expression Vector for Antibody, and Expression and Purification of the Antibody

Genes encoding the base sequences of heavy and light chains of the target antibody were obtained according to a method known to those skilled in the art using Assemble PCR and the like. Amino acid substitution was introduced using QuikChange Site-Directed Mutagenesis Kit (Stratagene), PCR, or the like according to a method known to those skilled in the art. Obtained plasmid fragments were inserted into expression vectors for animal cell to produce target heavy chain- and light chain-expression vectors. The base sequences of the obtained expression vectors were determined according to a method known to those skilled in the art. An antibody was expressed employing the following method. Each antibody expression vector, 15 μg, was cleaved with pvuI, and introduced into DG44 line (Invitrogen) by electroporation, which was seeded into a 96-well plate. After culturing in a CHO-S-SFM-II (Invitrogen) medium containing 500 μg/ml of G418 (Invitrogen), antibody production in the culture supernatant in a G418-resistance well was detected by ELISA. A clone observed to produce an antibody was further amplified, and the culture supernatant was collected and used for antibody purification. After cells were removed by centrifugation (approximately 2000 g, 5 minutes, room temperature), the culture supernatant was further passed through 0.22-μm filter MILLEX®-GV (Millipore). From the resultant culture supernatant, an antibody was purified using Hi Trap Protein G HP column (Amersham Biosciences #17-0404-01) according to a method known to those skilled in the art. The purified antibody was passed through a 0.22-μm filter (MILLIPORE #SLGV033RS) and used for analysis.

### INDUSTRIAL APPLICABILITY

An anti-ICAM3 antibody of the present invention is capable of exerting excellent proliferation-suppressing action and cytotoxic action on cells having ICAM3 expressed on surfaces thereof. Since exerting a strong action on various blood cancer cell lines, the anti-ICAM3 antibody of the present invention is particularly useful for treatment against leukemia, myeloma, malignant lymphoma, or the like. Moreover, because of the specificity, the anti-ICAM3 antibody of the present invention is applicable also to detection and screening of cells having ICAM3 on surfaces thereof.

### [Sequence Listing Free Text]

SEQ ID NO: 11

### <223> IB23_VH_FR1

SEQ ID NO: 12

### <223> IB23_VH_FR2

SEQ ID NO: 13

### <223> IB23_VH_FR3

SEQ ID NO: 14

### <223> IB23_VH_FR4

SEQ ID NO: 15

### <223> IB23_VH_CDR1

SEQ ID NO: 16

### <223> IB23_VH_CDR2

SEQ ID NO: 17

### <223> IB23_VH_CDR3

SEQ ID NO: 18

### <223> IB23_VL_FR1

SEQ ID NO: 19

### <223> IB23_VL_FR2

SEQ ID NO: 20

### <223> IB23_VL_FR3

SEQ ID NO: 21

### <223> IB23_VL_FR4

SEQ ID NO: 22

### <223> IB23_VL_CDR1

SEQ ID NO: 23

### <223> IB23_VL_CDR2

SEQ ID NO: 24

<223> IB23_VL_CDR3

1. An anti-ICAM3 antibody having a cytotoxic activity.

2. The anti-ICAM3 antibody according to claim 1, wherein
the cytotoxic activity is an ADCC activity.

3. The anti-ICAM3 antibody according to claim 1, which further has a cell-proliferation-suppressing activity.

4. The anti-ICAM3 antibody according to claim 1, which has an anti-tumor activity in vivo.

5. The anti-ICAM3 antibody according to claim 1, which does not substantially bind to ICAM1 and/or ICAM5.

6. An antibody of any one of (a) to (c) below:
(a) an antibody comprising heavy chain CDR1 having an amino acid sequence of SEQ ID NO: 15, heavy chain CDR2 having an amino acid sequence of SEQ ID NO: 16, heavy chain CDR3 having an amino acid sequence of SEQ ID NO: 17, light chain CDR1 having an amino acid sequence of SEQ ID NO: 22, CDR2 having an amino acid sequence of SEQ ID NO: 23, and light chain CDR3 having an amino acid sequence of SEQ ID NO: 24;
(b) an antibody of (a) in which one or more amino acids are substituted, deleted, added and/or inserted, the antibody of (b) having an activity equivalent to that of the antibody of (a); and
(c) an antibody which recognizes a same epitope as an epitope recognized by the antibody of any one of (a) and (b).

7. The anti-ICAM3 antibody according to claim 1, comprising a human-derived constant region.

8. A DNA encoding the antibody according to claim 1.

9. A pharmaceutical composition comprising the antibody according to claim 1 as an active ingredient.

10. The pharmaceutical composition according to claim 9, which is an anti-cancer agent.

11. The pharmaceutical composition according to claim 10, wherein the cancer is leukemia, myeloma, or malignant lymphoma.

12. A diagnosis method for a cancer, characterized by detecting any one of an ICAM3 protein and a gene encoding the ICAM3 protein.

13. The anti-ICAM3 antibody according to claim 2, which further has a cell-proliferation-suppressing activity.

14. The anti-ICAM3 antibody according to claim 6, comprising a human-derived constant region.

15. A DNA encoding the antibody according to claim 6.

16. A pharmaceutical composition comprising the antibody according to claim 6 as an active ingredient.


SEQUENCE LISTING (30 sequences)

SEQ ID NO: 1 - DNA, 1644 bp, Homo sapiens
  CDS (1)..(1641)
atggccaccatggtaccatccgtgttgtggcccagggcctgctggactctgctggtctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctctcagataacaggctcctctaacatcaccgtgtacaggctcccggagcgtgtggagctggcacccctgcctccttggcagccggtgggccagaacttcaccctgcgctgccaagtggaggatgggtcgccccggaccagcctcacggtggtgctgcttcgctgggaggaggagctgagccggcagcccgcagtggaggagccagcggaggtcactgccactgtgctggccagcagagacgaccacggagcccctttctcatgccgcacagaactggacatgcagccccaggggctgggactgttcgtgaacacctcagccccccgccagctccgaacctttgtcctgcccgtgacccccccgcgcctcgtggccccccggttcttggaggtggaaacgtcgtggccggtggactgcaccctagacgggctttttccagcctcagaggcccaggtctacctggcgctgggggaccagatgctgaatgcgacagtcatgaaccacggggacacgctaacggccacagccacagccacggcgcgcgcggatcaggagggtgcccgggagatcgtctgcaacgtgaccctagggggcgagagacgggaggcccgggagaacttgacggtctttagcttcctaggacccattgtgaacctcagcgagcccaccgcccatgaggggtccacagtgaccgtgagttgcatggctggggctcgagtccaggtcacgctggacggagttccggccgcggccccggggcagccagctcaacttcagctaaatgctaccgagagtgacgacggacgcagcttcttctgcagtgccactctcgaggtggacggcgagttcttgcacaggaacagtagcgtccagctgcgagtcctgtatggtcccaaaattgaccgagccacatgcccccagcacttgaaatggaaagataaaacgagacacgtcctgcagtgccaagccaggggcaacccgtaccccgagctgcggtgtttgaaggaaggctccagccgggaggtgccggtggggatcccgttcttcgtcaacgtaacacataatggtacttatcagtgccaagcgtccagctcacgaggcaaatacaccctggtcgtggtgatggacattgaggctgggagctcccactttgtccccgtcttcgtggcggtgttactgaccctgggcgtggtgactatcgtactggccttaatgtacgtcttcagggagcaccaacggagcggcagttaccatgttagggaggagagcacctatctgcccctcacgtctatgcagccgacagaagcaatgggggaagaaccgtccagagctgagtga

SEQ ID NO: 2 - protein, 547 aa, Homo sapiens
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 3 - DNA, 1599 bp, Homo sapiens
  CDS (1)..(1596)
atggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctga

SEQ ID NO: 4 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 5 - DNA, 2775 bp, Homo sapiens
  CDS (1)..(2772)
atgccagggccttcgccagggctgcgccgggcgctactcggcctctgggctgctctgggcctggggctcttcggcctctcagcggtctcgcaggagcccttctgggcggacctgcagcctcgcgtggcgttcgtggagcgcgggggctcgctgtggctgaattgcagcaccaactgccctcggccggagcgcggtggcctggagacctcgctgcgccgaaacgggacccagaggggtttgcgttggttggcgcggcagctggtggacattcgcgagccggagactcagcccgtctgcttcttccgctgcgcgcggcgcacactacaggcgcgtgggctcattcgcactttccagcgaccagatcgcgtagagctgatgccgctgcctccctggcagccggtgggcgagaacttcaccctgagctgtagggtccccggcgccgggccccgtgcgagcctcacgctgaccctgctgcggggcgcccaggagctgatccgccgcagcttcgccggtgaaccaccccgagcgcggggcgcggtgctcacagccacggtactggctcggagggaggaccatggagccaatttctcgtgtcgcgccgagctggacctgcggccgcacggactgggactgtttgaaaacagctcggcccccagagagctccgaaccttctccctgtctccggatgccccgcgcctcgctgctccccggctcttggaagttggctcggaaaggcccgtgagctgcactctggacggactgtttccagcctcagaggccagggtctacctcgcactgggggaccagaatctgagtcctgatgtcaccctcgaaggggacgcattcgtggccactgccacagccacagctagcgcagagcaggagggtgccaggcagctgatctgcaacgtcaccctggggggcgaaaaccgggagacccgggagaacgtgaccatctacagcttcccggcaccactcctgaccctgagcgaacccagcgtctccgaggggcagatggtgacagtaacctgcgcagctgggacccaagctctggtcacactggagggagttccagccgcggtcccggggcagcccgcccagcttcagctaaatgccaccgagaacgacgacagacgcagcttcttctgcgacgccaccctcgatgtggacggggagaccctgatcaagaacaggagcgcagagcttcgtgtcctatacgctccccggctagacgattcggactgccccaggagttggacgtggcccgagggcccagagcagacgctgcgctgcgaggcccgcgggaacccagaaccctcagtgcactgtgcgcgctccgacggcggggccgtgctggctctgggcctgctgggtccagtcactcgggcgctctcaggcacttaccgctgcaaggcggccaatgatcaaggcgaggcggtcaaggacgtaacgctaacggtggagtacgcaccagcgctggacagcgtgggctgcccagaacgcattacttggctggagggaacagaagcctcgctgagctgtgtggcgcacggggtaccgccgcctgatgtgatctgcgtgcgctctggagaactcggggccgtcatcgaggggctgttgcgtgtggcccgggagcatgcgggcacttaccgctgcgaagccaccaaccctcggggctctgcggccaaaaatgtggccgtcacggtggaatatggccccaggtttgaggagccgagctgccccagcaattggacatgggtggaaggatctgggcgcctgttttcctgtgaggtcgatgggaagccacagccaagcgtgaagtgcgtgggctccgggggcgccactgagggggtgctgctgccgctggcacccccagaccctagtcccagagctcccagaatccctagagtcctggcacccggtatctacgtctgcaacgccaccaaccgccacggctccgtggccaaaacagtcgtcgtgagcgcggagtcgccaccggagatggatgaatctacctgcccaagtcaccagacgtggctggaaggggctgaggcttccgcgctggcctgcgccgcccggggtcgcccttccccaggagtgcgctgctctcgggaaggcatcccatggcctgagcagcagcgcgtgtcccgagaggacgcgggcacttaccactgtgtggccaccaatgcgcatggcacggactcccggaccgtcactgtgggcgtggaataccggccagtggtggccgaacttgctgcctcgccccctggaggcgtgcgcccaggaggaaacttcacgttgacctgccgcgcggaggcctggcctccagcccagatcagctggcgcgcgcccccgggggccctcaacatcggcctgtcgagcaacaacagcacactgagcgtggcaggcgccatgggaagccacggcggcgagtacgagtgcgcacgcaccaacgcgcacgggcgccacgcgcggcgcatcacggtgcgcgtggccggtccgtggctatgggtcgccgtgggcggcgcggcggggggcgcggcgctgctggccgcgggggccggcctggccttctacgtgcagtccaccgcctgcaagaagggcgagtacaacgtgcaggaggccgagagctcaggcgaggccgtgtgtctcaacggagcgggcggcggcgctggcggggcggcaggcgcggagggcggacccgaggcggcggggggcgcggccgagtcgccggcggagggcgaggtcttcgccatacagctgacatcggcgtga

SEQ ID NO: 6 - protein, 924 aa, Homo sapiens
MPGPSPGLRRALLGLWAALGLGLFGLSAVSQEPFWADLQPRVAFVERGGSLWLNCSTNCPRPERGGLETSLRRNGTQRGLRWLARQLVDIREPETQPVCFFRCARRTLQARGLIRTFQRPDRVELMPLPPWQPVGENFTLSCRVPGAGPRASLTLTLLRGAQELIRRSFAGEPPRARGAVLTATVLARREDHGANFSCRAELDLRPHGLGLFENSSAPRELRTFSLSPDAPRLAAPRLLEVGSERPVSCTLDGLFPASEARVYLALGDQNLSPDVTLEGDAFVATATATASAEQEGARQLICNVTLGGENRETRENVTIYSFPAPLLTLSEPSVSEGQMVTVTCAAGTQALVTLEGVPAAVPGQPAQLQLNATENDDRRSFFCDATLDVDGETLIKNRSAELRVLYAPRLDDSDCPRSWTWPEGPEQTLRCEARGNPEPSVHCARSDGGAVLALGLLGPVTRALSGTYRCKAANDQGEAVKDVTLTVEYAPALDSVGCPERITWLEGTEASLSCVAHGVPPPDVICVRSGELGAVIEGLLRVAREHAGTYRCEATNPRGSAAKNVAVTVEYGPRFEEPSCPSNWTWVEGSGRLFSCEVDGKPQPSVKCVGSGGATEGVLLPLAPPDPSPRAPRIPRVLAPGIYVCNATNRHGSVAKTVVVSAESPPEMDESTCPSHQTWLEGAEASALACAARGRPSPGVRCSREGIPWPEQQRVSREDAGTYHCVATNAHGTDSRTVTVGVEYRPVVAELAASPPGGVRPGGNFTLTCRAEAWPPAQISWRAPPGALNIGLSSNNSTLSVAGAMGSHGGEYECARTNAHGRHARRITVRVAGPWLWVAVGGAAGGAALLAAGAGLAFYVQSTACKKGEYNVQEAESSGEAVCLNGAGGGAGGAAGAEGGPEAAGGAAESPAEGEVFAIQLTSA

SEQ ID NO: 7 - DNA, 411 bp, Mus musculus
  V_region (1)..(411)
  CDS (1)..(411)
atggaaaggcactggatctttctcttcctgttttcagtaactgcaggtgtccactcccaggtccagcttcagcagtctggggctgaactggcaaaacctggggcctcagtgaagatgtcctgcaaggcttctggctacacctttactacctactggatgcactgggtaaaacagaggcctggacagggtctggaatggattggatacattaatcctaacactgattatactgaatacaatcagaagttcaaggacaaggccacattgactgcagacaagtcctccagcacagccttcatgcaactgagcagcctgacatctgaggactctgcagtctattactgtgcaagctcgagggatgcttaccacgggacttactggggccaaggaactctggtcactgtctctgca

SEQ ID NO: 8 - protein, 137 aa, Mus musculus
MERHWIFLFLFSVTAGVHSQVQLQQSGAELAKPGASVKMSCKASGYTFTTYWMHWVKQRPGQGLEWIGYINPNTDYTEYNQKFKDKATLTADKSSSTAFMQLSSLTSEDSAVYYCASSRDAYHGTYWGQGTLVTVSA

SEQ ID NO: 9 - DNA, 381 bp, Mus musculus
  V_region (1)..(411)
  CDS (1)..(381)
atggtgtccacttctcagctccttggacttttgcttttctggacttcagcctccagatgtgacattgtgatgactcagtctccagccaccctgtctgtgactccaggagatagagtctctctttcctgcagggccagtcagagtattagcgactacttacactggtatcaacagaaatcacatgagtctccaaggcttctcatcagatatgcttcccaatccatctctgggatcccctccaggttcagtggcagtggatcagggtcagatttcactctcagtatcaacaatgtagaacctgaagatgttggagtgtattactgtcaaaatggtcacaactttccgctcacgttcggtgctgggaccaagctggagctgaaa

SEQ ID NO: 10 - protein, 127 aa, Mus musculus
MVSTSQLLGLLLFWTSASRCDIVMTQSPATLSVTPGDRVSLSCRASQSISDYLHWYQQKSHESPRLLIRYASQSISGIPSRFSGSGSGSDFTLSINNVEPEDVGVYYCQNGHNFPLTFGAGTKLELK

SEQ ID NO: 11 - protein, 30 aa, Mus musculus
  MISC_FEATURE (1)..(30): IB23_VH_FR1
QVQLQQSGAELAKPGASVKMSCKASGYTFT

SEQ ID NO: 12 - protein, 14 aa, Mus musculus
  MISC_FEATURE (1)..(14): IB23_VH_FR2
WVKQRPGQGLEWIG

SEQ ID NO: 13 - protein, 32 aa, Mus musculus
  MISC_FEATURE (1)..(32): IB23_VH_FR3
KATLTADKSSSTAFMQLSSLTSEDSAVYYCAS

SEQ ID NO: 14 - protein, 11 aa, Mus musculus
  MISC_FEATURE (1)..(11): IB23_VH_FR4
WGQGTLVTVSA

SEQ ID NO: 15 - protein, 5 aa, Mus musculus
  MISC_FEATURE (1)..(5): IB23_VH_CDR1
TYWMH

SEQ ID NO: 16 - protein, 17 aa, Mus musculus
  MISC_FEATURE (1)..(17): IB23_VH_CDR2
YINPNTDYTEYNQKFKD

SEQ ID NO: 17 - protein, 9 aa, Mus musculus
  MISC_FEATURE (1)..(9): IB23_VH_CDR3
SRDAYHGTY

SEQ ID NO: 18 - protein, 23 aa, Mus musculus
  MISC_FEATURE (1)..(23): IB23_VL_FR1
DIVMTQSPATLSVTPGDRVSLSC

SEQ ID NO: 19 - protein, 15 aa, Mus musculus
  MISC_FEATURE (1)..(15): IB23_VL_FR2
WYQQKSHESPRLLIR

SEQ ID NO: 20 - protein, 32 aa, Mus musculus
  MISC_FEATURE (1)..(32): IB23_VL_FR3
GIPSRFSGSGSGSDFTLSINNVEPEDVGVYYC

SEQ ID NO: 21 - protein, 10 aa, Mus musculus
  MISC_FEATURE (1)..(10): IB23_VL_FR4
FGAGTKLELK

SEQ ID NO: 22 - protein, 11 aa, Mus musculus
  MISC_FEATURE (1)..(11): IB23_VL_CDR1
RASQSISDYLH

SEQ ID NO: 23 - protein, 7 aa, Mus musculus
  MISC_FEATURE (1)..(7): IB23_VL_CDR2
YASQSIS

SEQ ID NO: 24 - protein, 9 aa, Mus musculus
  MISC_FEATURE (1)..(9): IB23_VL_CDR3
QNGHNFPLT

SEQ ID NO: 25 - DNA, 996 bp, Homo sapiens
  C_region (1)..(996)
  CDS (1)..(990)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 26 - protein, 330 aa, Homo sapiens [sequence omitted]

SEQ ID NO: 27 - DNA, 327 bp, Homo sapiens
  C_region (1)..(327)
  CDS (1)..(321)
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgataa

SEQ ID NO: 28 - protein, 107 aa, Homo sapiens [sequence omitted]

SEQ ID NO: 29 - DNA, 996 bp, Homo sapiens
  C_region (1)..(996)
  CDS (1)..(990)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 30 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK